(19) United States
(12) Patent Application Publication
(10) Pub. No.: US 2022/0054606 A1
(43) Pub. Date: Feb. 24, 2022

(54) ASPARAGINASE-INDUCED GLUTAMINE DEPLETION COMBINED WITH BCL-2 INHIBITION FOR TREATMENT OF HEMATOLOGIC AND SOLID CANCERS

(71) Applicants: Ashkan EMADI, Clarksville, MD (US); Rena G. LAPIDUS, Baltimore, MD (US)

(72) Inventors: Ashkan EMADI, Clarksville, MD (US); Rena G. LAPIDUS, Baltimore, MD (US)

(73) Assignee: UNIVERSITY OF MARYLAND, BALTIMORE, Baltimore, MD (US)

(21) Appl. No.: 17/415,344

(22) PCT Filed: Dec. 19, 2019
(86) PCT No.: PCT/US2019/067374

§ 371 Date: Jun. 17, 2021

Related U.S. Application Data
(60) Provisional application No. 62/781,798, filed on Dec. 19, 2018.

(51) Int. Cl. A61K 38/50; A61K 31/635; A61K 31/5377; A61K 45/06; A61P 35/02; C12N 9/82

(52) U.S. Cl. CPC A61K 38/50; A61K 31/635; A61K 31/5377; C12Y 305/01001; A61P 35/02; C12N 9/82; A61K 45/06

(57) ABSTRACT

Methods of treating cancer and prolonging survival of a subject having cancer, such as acute myeloid leukemia, via administration of therapeutically effective amounts of agents that depletes plasma glutamine and agents that inhibit BCL-2 activity are detailed herein. Suitable agents that depletes plasma glutamine include asparaginase Suitable agents that inhibit BCL-2 activity include Venetoclax.

### BACKGROUND OF INVENTION

The current lifetime risk of a human developing acute myeloid leukemia (AML) in the United States is approximately 0.5%, which means that about 1 in 250 men and women born in the United States today will be diagnosed with AML during their lifetime. With approximately 73% of subjects diagnosed with AML succumbing to the disease within five years, the disease is particularly lethal. Patients with relapsed/refractory (R/R) AML have even more dismal outcome with a 3-year overall survival (OS) rate <10%. Treatment options for AML, especially in the R/R setting, have not changed significantly over the last few decades, and remains an area of unmet need.

Venetoclax (Ven) is an orally bioavailable small molecule that specifically inhibits binding of BIM (BCL-2-like protein 11) and BAX proteins to BCL-2, resulting in activation of the pro-apoptotic protein BAK, which triggers apoptosis via mitochondrial outer membrane permeabilization and activation of caspases [1]. In 2018, Ven, in combination with a DNA methyltransferase inhibitor (DNMTI), azacitidine or decitabine, or low-dose cytarabine was approved by the Food and Drug Administration (FDA) for the treatment of newly diagnosed AML in adults who are age 75 years or older, or who have comorbidities that preclude use of intensive induction chemotherapy [2, 3]. While results of Ven in combination with DNMTIs for newly diagnosed AML patients are encouraging, Ven is less effective in patients with R/R AML [4], either as a monotherapy [5] or in combination with DNMTIs [6], confirming the inherent resistance to Ven and underscoring the importance of developing novel rational combination therapies.

Targeting glutamine metabolic pathways can overcome Ven resistance [7, 8]. AML cells are sensitive to extracellular glutamine depletion or manipulation of intracellular glutamine metabolism [9, 10]. L-asparaginase, long a standard in treatment of acute lymphoblastic leukemia (ALL), converts asparagine and glutamine to aspartate and glutamate, respectively, decreasing plasma concentrations of asparagine and glutamine [11]. Approved clinical asparaginases are isolated from either E. coli (e.g. pegaspargase) or Erwinia chrysanthemi (crisantaspase); the latter having higher glutaminase activity [12, 13]. In a clinical study, crisantaspase produced complete plasma glutamine depletion in patients, associated with anti-leukemic activity in R/R AML, and no dose-limiting toxicity [14]. Long acting crisantaspase, Pegcrisantaspase (PegC), is a recombinant pegylated Erwinia asparaginase that has been tested in pediatric patients with ALL [15].

Combining the activities of Ven and an asparaginase might prove to be useful in the treatment of AML. The present invention is directed to such combinations for use in treating disease such as AML.

### SUMMARY OF INVENTION

Provided herein are combinations of (i) agents that deplete plasma glutamine and (ii) agents that inhibit BCL-2 activity. These combinations can be used in the treatment of cancers, such as leukemia, and in prolonging survival of subjects having cancer.

In a first embodiment, the present invention is drawn to methods of treating cancer in a subject, comprising administering therapeutically effective amounts of a first agent that depletes plasma glutamine and a second agent that inhibits BCL-2 activity to a subject having cancer. The first and second agents may be administered in either order, alone or in combination, sequentially or concurrently, with overlapping or non-overlapping periods of administration.

In a second embodiment, the present invention is drawn to methods of treating cancer in a subject, comprising concurrently administering therapeutically effective amounts of a first agent that depletes plasma glutamine and a second agent that inhibits BCL-2 activity to a subject having cancer. The first and second agents may be administered together or separately, with partially overlapping or fully overlapping periods of administration.

In a third embodiment, the present invention is drawn to methods of treating cancer in a subject, comprising sequentially administering therapeutically effective amounts of a first agent that depletes plasma glutamine and a second agent that inhibits BCL-2 activity to a subject having cancer. The first and second agents may be administered with partially overlapping or non-overlapping periods of administration.

As suggested above, the combinations of agents provided herein can also be used in methods of prolonging survival of subjects having cancer. Therefore, and in a fourth embodiment, the present invention is drawn to methods of prolonging survival of a subject having cancer, comprising administering therapeutically effective amounts of a first agent that depletes plasma glutamine and a second agent that inhibits BCL-2 activity to a subject having cancer. The first and second agents may be administered in either order, alone or in combination, sequentially or concurrently, with overlapping or non-overlapping periods of administration.

In a fifth embodiment, the present invention is drawn to methods of prolonging survival of a subject having cancer, comprising concurrently administering therapeutically effective amounts of a first agent that depletes plasma glutamine and a second agent that inhibits BCL-2 activity to a subject having cancer. The first and second agents may be administered together or separately, with partially overlapping or fully overlapping periods of administration.

In a sixth embodiment, the present invention is drawn to methods of prolonging survival of a subject having cancer, comprising sequentially administering therapeutically effective amounts of a first agent that depletes plasma glutamine and a second agent that inhibits BCL-2 activity to a subject having cancer. The first and second agents may be administered with partially overlapping or non-overlapping periods of administration.

In each embodiment and aspect of the invention, the agent that depletes plasma glutamine may be, but is not limited to, an asparaginase. Suitable examples of asparaginases that may be used in the methods of the invention include, but are not limited to, E. coli-derived short acting asparaginase, polyethylene glycosylated E. coli-derived asparaginase (pegaspargase and calaspargase pegol-mknl), Erwinia chrysanthemi (recently termed Dickeya dadantii)-derived short acting asparaginase (Erwinaze and recombinant crisantaspase using Pseudomonas fluorescens expression platform), polyethylene glycosylated Erwinia chrysanthemi-derived asparaginase (pegcrisantaspase; PegC). Other examples of asparaginase that may be used in the methods of the invention are asparaginase enzymes isolated from other bacteria (e.g. Coliform bacteria, Pseudomonas aeruginosa, Pectobacterium caratovorum, Bacillus subtilis, Serratia marcescens, Staphylococcus capitis), fungi (e.g. Fusarium equiseti, Aspergillus terreus, Aspergillus nieger), actinomycetes (e.g. Streptomyces albidoflavus, Marine Streptomycete strain), and plants (e.g. Soyabean leaves, Ocimum sanctum L, Withania somnifera, Soyabean seeds, Pisum sativum).

In each embodiment and aspect of the invention, the agent that inhibits BCL-2 activity may be, but is not limited to, one or more of venetoclax (Ven), BCL201, and/or navitoclax.

In each embodiment and aspect of the invention, the cancer may be, but is not limited to, a cancer overexpressing a BCL-2 family member or a cancer overexpressing a kinase or having an increased or constitutively active kinase (e.g., tyrosine kinases, serine/threonine kinases). Examples of cancers exhibiting overexpression of BCL-2 include, but are not limited to, hematologic malignancies (including leukemia, lymphoma, and multiple myeloma) and solid tumors (including prostate, breast, small cell and non-small cell lung cancers, ovarian, neuroblastoma, bladder, colorectal, and head and neck cancers). Examples of cancers overexpressing a kinase or having an increased or constitutively active kinase include, but are not limited to, hematologic malignancies (including leukemia, lymphoma, and multiple myeloma) and solid tumors (including prostate, breast, small cell and non-small cell lung cancers, ovarian, neuroblastoma, bladder, colorectal, mesothelioma, and head and neck cancers).

In each embodiment and aspect of the invention, the cancer may be, but is not limited to, one or more of acute myeloid leukemia (AML), complex karyotype acute myeloid leukemia (CK-AML), acute lymphoblastic leukemia (ALL), B cell ALL (B-ALL), T cell ALL (T-ALL), chronic myeloid leukemia (CML), chronic lymphoid leukemia (CLL); lymphoma (including B cell and T cell); myeloma; myelodysplastic syndrome; non-small cell lung cancer; pancreatic cancer; gastric cancer; Kaposi's sarcoma; hepatocellular carcinoma; osteosarcoma; laryngeal squamous cell carcinoma; metastatic uveal melanoma; lung and splenic metastases; advanced non-small cell lung cancer; cervical carcinoma; colorectal cancer; breast cancer; prostate cancer; mesothelioma; and all other hematologic malignancies and solid cancers including brain cancers.

In each embodiment and aspect of the invention, the first and second agents may be independently formulated in pharmaceutical compositions comprising one of the agents, or combinations of two, three or more of the agents, and a pharmaceutically acceptable carrier or diluent.

In each embodiment and aspect of the invention, the first and second agents may be administered via the same or different modes of administration, and combinations thereof when there are more than two agents being administered to a subject.

In certain aspects of each embodiment, the combination of the first and second agent has an additive therapeutic effect on the cancer. In other aspects of each embodiment, the combination of the first and second agents has a synergistic therapeutic effect on the cancer.

In certain aspects of each embodiment, the first and second agents are administered to the subject in the same pharmaceutical composition and via the same mode of administration.

In a particular aspect, the invention is directed to a method of treating AML in a subject, comprising administering therapeutically effective amounts of an asparaginase and Ven to a subject having AML. The asparaginase and Ven may be administered in either order, alone or in combination, sequentially or concurrently, with overlapping or non-overlapping periods of administration. In one aspect of the method, the asparaginase is PegC.

In a particular aspect, the invention is directed to a method of treating AML in a subject, comprising concurrently administering therapeutically effective amounts of an asparaginase and Ven to a subject having AML. The asparaginase and Ven may be administered together or separately, with partially overlapping or fully overlapping periods of administration. In one aspect of the method, the asparaginase is PegC.

In a particular aspect, the invention is directed to a method of treating AML in a subject, comprising sequentially administering therapeutically effective amounts of an asparaginase and Ven to a subject having AML. The asparaginase and Ven may be administered with partially overlapping or non-overlapping periods of administration. In one aspect of the method, the asparaginase is PegC.

In a particular aspect, the invention is directed to a method of prolonging survival of a subject having AML, comprising administering therapeutically effective amounts of an asparaginase and Ven to a subject having AML. The asparaginase and Ven may be administered in either order, alone or in combination, sequentially or concurrently, with overlapping or non-overlapping periods of administration. In one aspect of the method, the asparaginase is PegC.

In a particular aspect, the invention is directed to a method of prolonging survival of a subject having AML, comprising concurrently administering therapeutically effective amounts of an asparaginase and Ven to a subject having AML. The asparaginase and Ven may be administered together or separately, with partially overlapping or fully overlapping periods of administration. In one aspect of the method, the asparaginase is PegC.

In a particular aspect, the invention is directed to a method of prolonging survival of a subject having AML, comprising sequentially administering therapeutically effective amounts of an asparaginase and Ven to a subject having AML. The asparaginase and Ven may be administered with partially overlapping or non-overlapping periods of administration. In one aspect of the method, the asparaginase is PegC.

In each embodiment and aspect of the invention, the therapeutically effective amount of the agent that depletes plasma glutamine varies based on body weight or body surface area of the subject. When the agent that depletes plasma glutamine is an asparaginase, the therapeutically effective amount of the agent may be between about 10 and about 50,000 IU/m².

In each embodiment and aspect of the invention, the therapeutically effective amount of the agent that inhibits BCL-2 activity varies based on pharmacologic characteristics of the agent including drug-drug interaction and food effect. As a non-limiting example, the therapeutically effective amount of the agent that inhibits BCL-2 activity may be between about 10 mg and about 800 mg.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits of the invention.

### BRIEF DESCRIPTION OF DRAWINGS

FIG. 1. In vitro anti-AML activity of the combination of Ven and PegC. (A) PegC potentiates Ven's anti-AML effect in two human AML cell lines, MOLM-14 and MonoMac6. Cells were exposed for 72 hours (h) to Ven at a range of concentrations with vehicle (DMSO) or 0.01, 0.006 or 0.001 IU/mL PegC. After WST-1 termination, IC₅₀ values were calculated by Graphpad Prism. (B) MOLM-14 and MonoMac6 cells were exposed to Ven (left columns) at a range of concentrations with PegC (right columns) at a fixed concentration of 0.001 IU/mL (IC₁₀) for 72 h. Cells were counted with trypan blue exclusion. (C) Primary AML cells, AML29 and AML31, were plated overnight then treated with vehicle, Ven, PegC, or Ven-PegC. Cytotoxicity was assessed at 24 h and 48 h by lactate dehydrogenase (LDH) release as an indicator of cell death using the Cytotox 96 Cytotoxicity Assay. (D) MOLM-14 cells were plated overnight then treated with vehicle, Ven (5.2 μM; second column), PegC (0.025 IU/mL; third column), or Ven-PegC (fourth column). At 48h, cells were fixed in ethanol, stained with propidium iodide (PI), then analyzed by flow cytometry. (E) MOLM-14 cells were treated with Ven (third column), PegC (second column) or their combination (fourth column) at corresponding IC_{50S} for 24 h. Cell lysates were prepared, followed by western blot analysis for caspase 3.

FIG. 2. Alteration of gene transcription in AML after treatment with Ven, PegC, and Ven-PegC. (A) Principal component analysis (PCA) of whole-transcriptome RNA-seq data from the indicated treatment. (B) Mean expression profile of MOLM-14 transcripts upon treatment with Ven (5.2 μM) and/or PegC (0.025 IU/mL) for 16 h. DMSO was used as control. Unregulated genes are shown, ranked by log 2 fold-change (FC). (C) Bar plots showing the number of genes significantly altered (green (above zero) upregulated, pink (below zero) downregulated) compared with cells treated with vehicle control. Protein coding transcripts were a predominant feature detected in all treatment conditions. (D) Venn diagram representing the number of differentially expressed genes (DEG) between Ven-, PegC-, and Ven-PegC-treated samples. (E) qRT-PCR analysis of p90RSK expression in MOLM-14 cells treated with control (first column), Ven (second column), PegC (third column) and Ven-PegC (fourth column). Results were normalized to control-treated cells and expressed as mean±SD (n=3). Statistical analysis was performed using one-way ANOVA, and p-values were adjusted using Bonferroni's correction method *p<0.05, **p<0.01, ***p<0.005 vs control-treated corresponding cells, ^{a}p<0.05, ^{b}p<0.01 vs Ven-PegC treated corresponding cells.

FIG. 3. Ven-PegC impedes the MAPK/ERK and mTOR pathways, depleting cap-dependent translation. (A, B and D) MOLM-14 cells were treated with PegC (second column), Ven (third column) and Ven-PegC (fourth column) for 16 h and lysed, and cellular lysates were probed with the indicated antibodies. The bar diagram represents densitometric quantification of three independent experiments. (C) Precleared cellular lysates of MOLM-14 cells treated with PegC, Ven and Ven-PegC were incubated with m⁷GTP sepharose beads for 2 h, followed by washing and probing with the indicated antibodies. The bar diagram represents densitometric quantification of three independent experiments. Results were normalized to corresponding control-treated cells and expressed as mean±SD (n=3). Statistical analysis was performed using one-way ANOVA, and p-values were adjusted using Bonferroni's correction method *p<0.05, **p<0.01, ***p<0.005 vs corresponding control-treated cells, ^{a}p<0.05, ^{b}p<0.01, ^{c}p<0.005 vs corresponding Ven-PegC-treated cells.

FIG. 4. Polysomal profiling reveals robust inhibition of active translation. (A) Heatmap showing unsupervised clustering of the differential bound genes (DBGs) in the polysome fraction. (B) Heat map defining translation efficiency (TE) and transcription and translation log 2-transformed fold-change of genes expressed as z-scores in indicated comparisons to the control group. (C) Bar diagram indicates the differential expression of genes with each treatment (green (above zero) upregulated, pink (below zero) downregulated). (D) Venn diagram showing overlap between polysome Ribo-seq annotated open reading frames (ORFs) in the indicated conditions.

FIG. 5. Ven-PegC was well tolerated in NRG mice. (A) NRG mice were dosed with 1000 IU/kg or 500 IU/kg PegC IV once per week for two weeks. (B) NRG mice were dosed with 250 IU/kg PegC IV weekly for two weeks alone or in combination with 100 mg/kg Ven dosed PO 5 days per week for two weeks. In both studies, mice were observed 5 days per week and monitored for an additional four weeks post dosing. (C) The maximum tolerated dose (MTD) of Ven-PegC combination was determined by showing no significant weight loss.

FIG. 6. Efficacy of Ven, PegC and Ven-PegC combination in an orthotopic patient-derived xenograft (PDX) model of relapsed AML with complex karyotype. AML45-luc (1×10⁶ cells) were injected IV into NRG mice, and after confirmation of engraftment, mice were treated with vehicle, Ven, PegC, or Ven-PegC. Ven was dosed at 75 mg/Kg once daily on days 1-5, 8-12, 23-24, 30-33, 78-82, 86-89, 99-103. PegC was dosed at 200 IU/Kg weekly on days 1, 8, 23, 30, 78, 87, 99. No treatment was administrated between Week 5 and Week 11. One mouse in the Ven-PegC group died in the second week due to a technical error. Mice were imaged weekly. (A) Imaging at serial time points. (B) Photon intensity versus time. Photon intensity correlates with AML burden. Each line is a different mouse. (C) Kaplan-Meier survival curve of mice treated with vehicle, Ven, PegC or their combination. Mice that died for reasons other than leukemia burden (Photon intensity <1.5×10⁸) were censored. (D) Percent weight changes versus time. Mice recovered after initial weight loss, and all gained weight in long term.

FIG. 7. Efficacy of single agents Ven, PegC, azacitidine (Aza), and combination of Aza-Ven and Ven-PegC in U937-luc cells. U937-luc cells (0.25×10⁶) were injected IV into NRG mice. Following engraftment, mice were treated with PegC (200 IU/kg IV weekly) and/or Ven (75 mg/kg PO 5 days weekly) and/or azacitidine (0.5 mg/kg subcutaneously 5 days weekly). Mice were imaged weekly and survival was monitored. Ven was dosed on days 1-5, 8-10, PegC on days 1, 8, and azacitidine on days 2-5, 8-12. In the Ven-PegC group (the only mice to live beyond day 18), mice were treated with one more dose of PegC and 3 more doses of Ven. (A) Imaging at serial time points. (B) Photon intensity versus time. Photon intensity correlates with AML burden. Each symbol represents a different mouse. (C) Kaplan-Meier survival curve of mice treated with vehicle, Ven, PegC or their combination, Aza and Aza-Ven.

FIG. 8. Pharmacodynamic (PD) effects of Ven-PegC in vivo in AML45-luc. AML45-luc (1×10⁶) cells were injected IV into NRG mice. After confirmation of engraftment by imaging, mice were treated with vehicle, Ven, PegC or Ven-PegC. Mice were dosed with PegC on days 1, 8, 15, 29 and 36, and Ven on days 1-5, 8-12, 15-19, 29-33 and 36-38. Mice were imaged on day 33 and euthanized on day 39. (A) Imaging on Days 1 and 33. (B) Photon intensity versus time. Each line represents a different mouse. (C) Glutamine, Asparagine, and Glutamate levels (micromolar; μM) levels in plasma from mice euthanized post last dose of treatments. (D) Western blot analysis of bone marrow lysates from mice treated with vehicle, Ven, PegC and Ven-PegC. (E) Bar diagram represents the densitometry quantification for three independent experiments (control×first column; Ven×first column; PegC×third column; Ven-PegC×fourth column). Results were normalized with Control-treated corresponding cells and expressed as mean±SD (n=3). Statistical analysis was performed using one-way ANOVA, and p-values were adjusted using Bonferroni's correction method *p<0.05, **p<0.01, ***p<0.005 vs Control treated corresponding cells, ^{a}p<0.05, ^{b}p<0.01, ^{c}p<0.005 vs Ven-PegC treated corresponding cells.

FIG. 9. Kaplan-Meier survival curve of AML45-luc mice died due to any cause. A sensitivity analysis with outcome as death due to any cause in mice treated with vehicle, Ven, PegC or their combination.

FIG. 10. Plasma glutamine levels in five patients enrolled in a clinical trial of single agent crisantaspase (Erwinaze) for treatment of relapsed or refractory AML. Patients received short acting Erwinia asparaginase on Days 1, 3, 5, 8, 10, and 12. Median trough plasma glutamine level 48 h after initial Erwinaze administration was 27.6 (range <12.5-227) μtmol/L. Four of 5 patients (80%, lower limit of 1-sided 95% CI: 34%) achieved at least one nadir glutamine value <120 μmol/L. The fold reduction (FR) in glutamine level at 3 days, relative to baseline, was 0.16 (p<0.001 for rejecting FR=1). With the use of short acting asparaginase, glutamine levels increased significantly on day 8 (72 h trough) as compared to day 5 (p<0.001).

### DETAILED DESCRIPTION OF THE INVENTION

### I. DEFINITIONS

Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### II. THE PRESENT INVENTION

Acute myeloid leukemia (AML) is a devastating illness, with 73% of diagnosed subjects succumbing to the disease within five years. Subsets of the disease can be even more difficult to treat. For example, complex karyotype acute myeloid leukemia (CK-AML), defined as harboring three or more unrelated chromosome abnormalities, comprises 10-12% of AML cases, the second largest cytogenetic subset in patients with AML [37], and has a dismal outcome even with aggressive treatments including intensive chemotherapy, DNA methyltransferase inhibitors (DNMTIs) and allogeneic hematopoietic stem cell transplantation [38,39].

Although, venetoclax (Ven) combined with DNMTIs is promising for frontline AML, single agent Ven resulted in only 19% overall response rate with a median time to AML progression of 2.5 months [5]. In the relapsed setting, treatment with Ven in combination with low-intensity chemotherapy resulted in an equally poor 21% overall objective response rate in AML with half of the responses being CR with or without blood count recovery [6]. A major cause of poor response to Ven is the overexpression of other anti-apoptotic proteins [40,41]. Among these proteins, MCL-1 overexpression has a key role in mechanism of resistance to BCL-2 inhibition in general [42,43] and to Ven in particular [44]. Hence, the development of combination therapy comprising Ven and an agent with novel mechanism of action that can directly or indirectly inhibit MCL-1 and overcome Ven resistance is required. While first-in-human clinical trials of single agent MCL-1 inhibitors (e.g. NCT03465540) are ongoing, the adverse event profile of these agents is under investigation. Combination of BCL-2 and MCL-1 inhibitors seems to have substantial toxicity to normal tissues including hematopoietic stem cells [45], hepatocytes [46] and cardiomyocytes [47]. Therefore indirect induction of MCL-1 downregulation might be a viable strategy to circumvent Ven resistance in the clinic.

mRNA and protein expression of glutamate-ammonia ligase, also called glutamine synthetase, was markedly upregulated in a Ven-resistant cell line, compared to its Ven-sensitive counterpart [44]. Glutamine synthetase (encoded by GLUL) promotes cell survival and proliferation in cancer cells [48]. Pharmacologic inhibition of glutaminase, an enzyme that converts glutamine to glutamate intracellularly, was reported to sensitize AML cells to Ven [7,49]. MCL-1 protein has an extremely short half-life, and maintenance of cellular MCL-1 protein levels is dependent on active cap-mRNA translation and is a target of phosphorylated eukaryotic translation initiation factor 4E (eIF4E) [50]. Asparaginase-induced inhibition of eIF4E-binding protein 1 (4E-BP1) phosphorylation, necessary for maintenance of active cap-mRNA translation, has been reported to decrease MCL-1 expression [9].

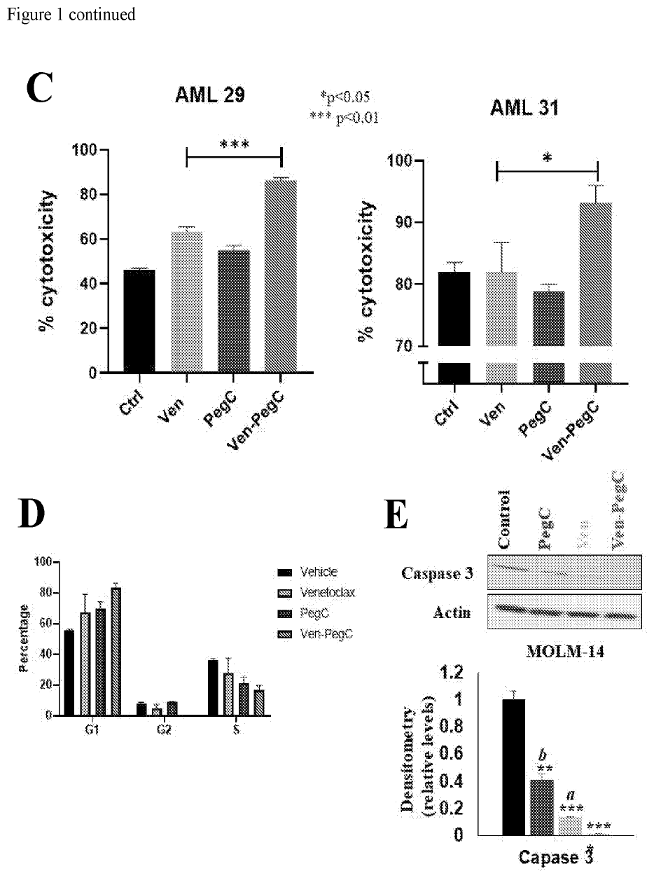

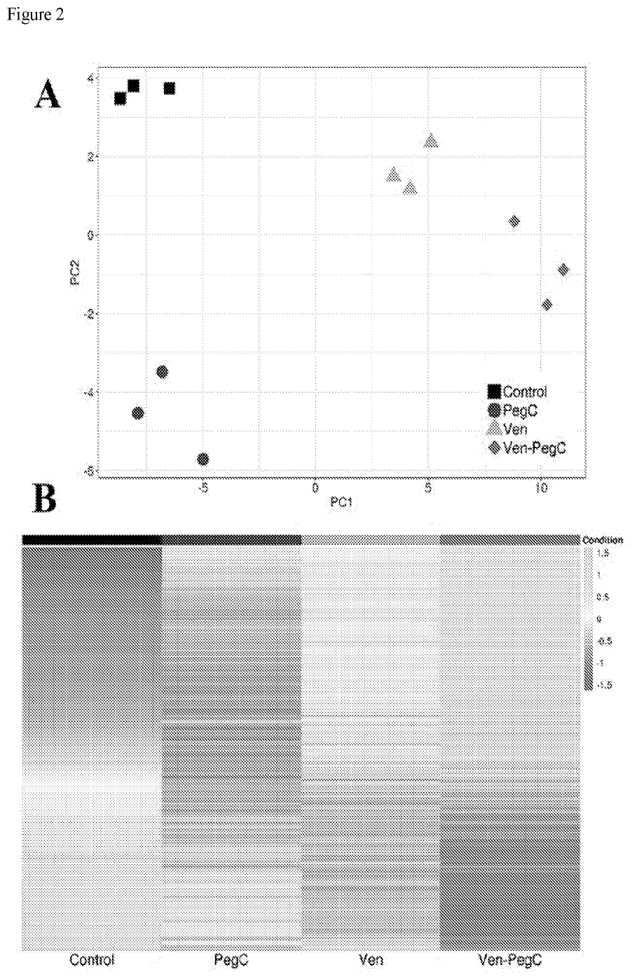

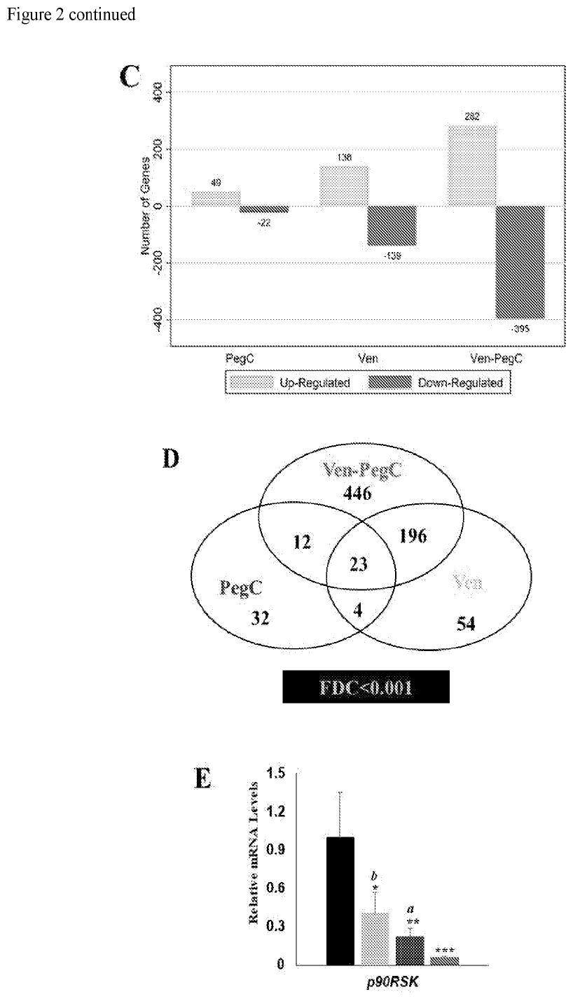

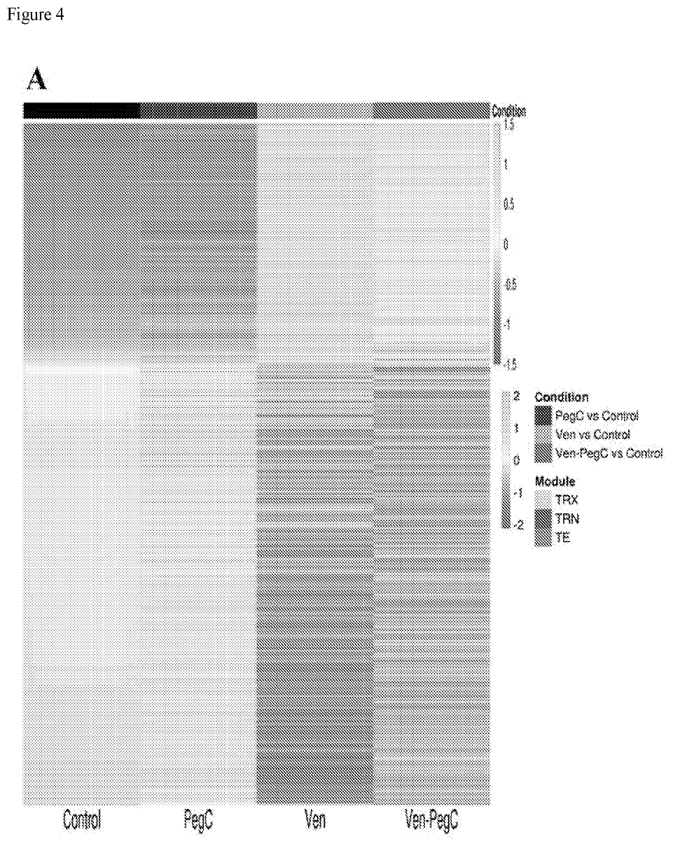

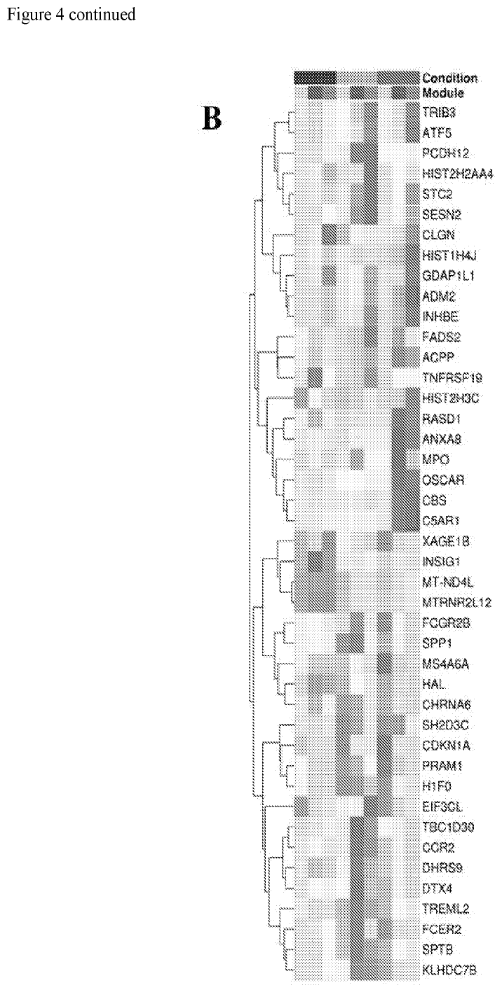

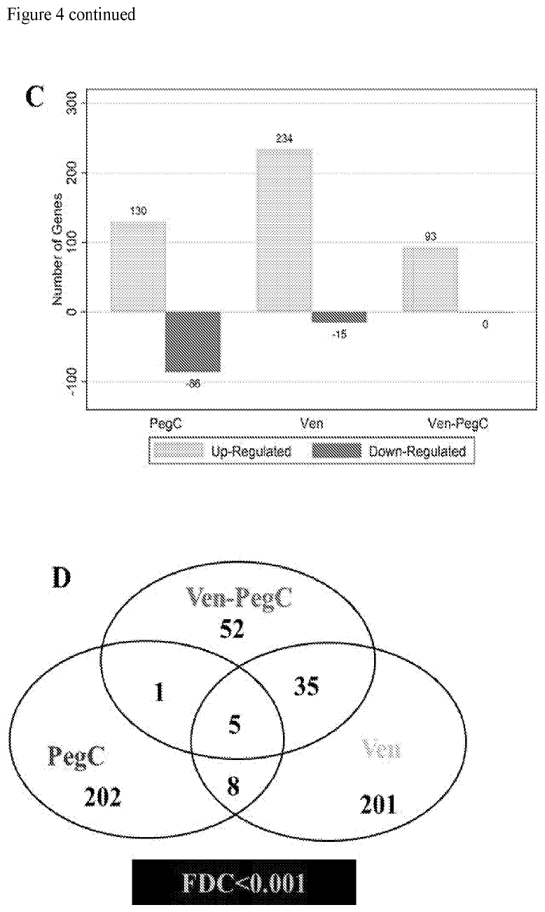

As discussed in detail herein, the inventors discovered that PegC-mediated glutamine deprivation can synergize with Ven against AMLs via downregulation of MCL-1 synthesis. Treatment with asparaginase enzymes is one of the most effective and clinically applicable ways to interfere with glutamine metabolism. It has been shown that asparaginase crisantaspase isolated from bacteria Erwinia chrysantemi can completely deplete plasma glutamine in patients with AML and can provide clinical benefit [14]. The Examples presented herein focus on combining long-acting crisantaspase (PegC) with Ven at clinically relevant concentrations. The experiments demonstrate that Ven is a potent DNA transcriptional modulator (FIG. 2), whereas PegC is a potent mRNA translational modulator (FIG. 4). Together, Ven-PegC diminished p90RSK expression and augmented eIF4E-4EBP1 interaction on cap-binding complexes, resulting in inhibition of cap-dependent translation and MCL-1 expression. This strong interference with cellular protein synthesis resulted in an effective AML cell killing in vitro [51]. Although PegC had modest anti-AML activity as monotherapy, it effectively synergized with Ven and sensitized inherently resistant human AML cells in vitro.

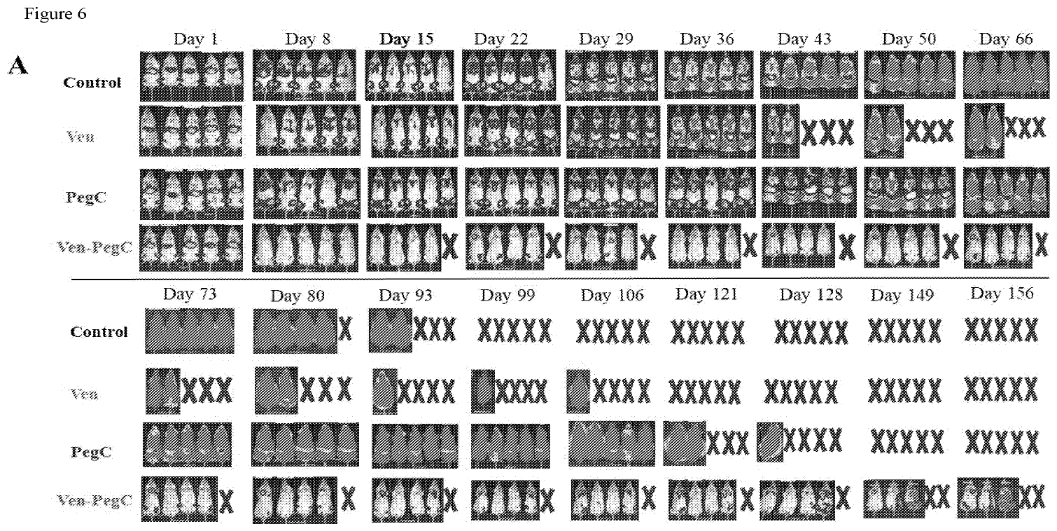

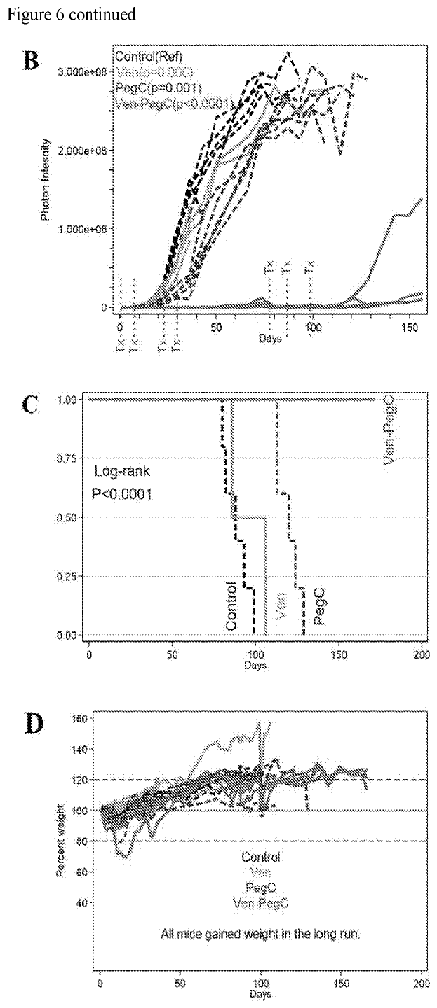

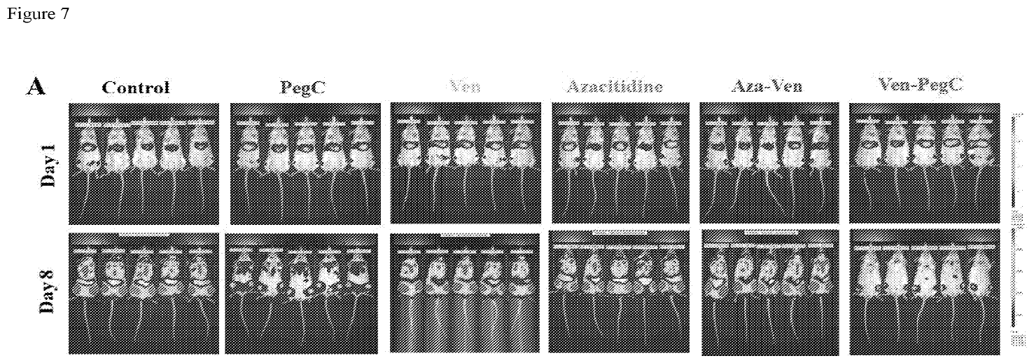

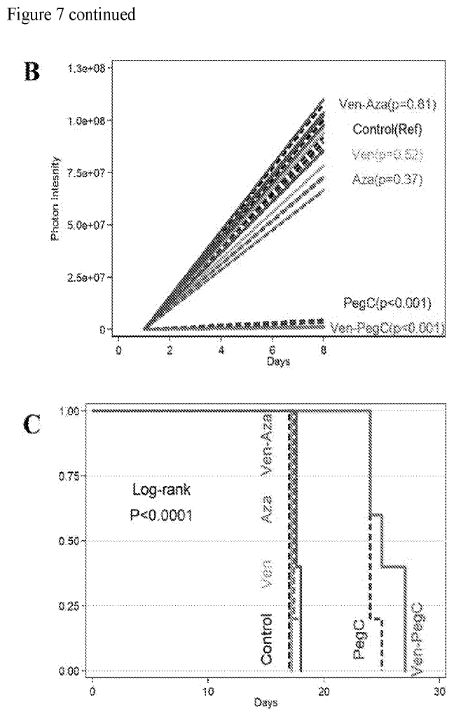

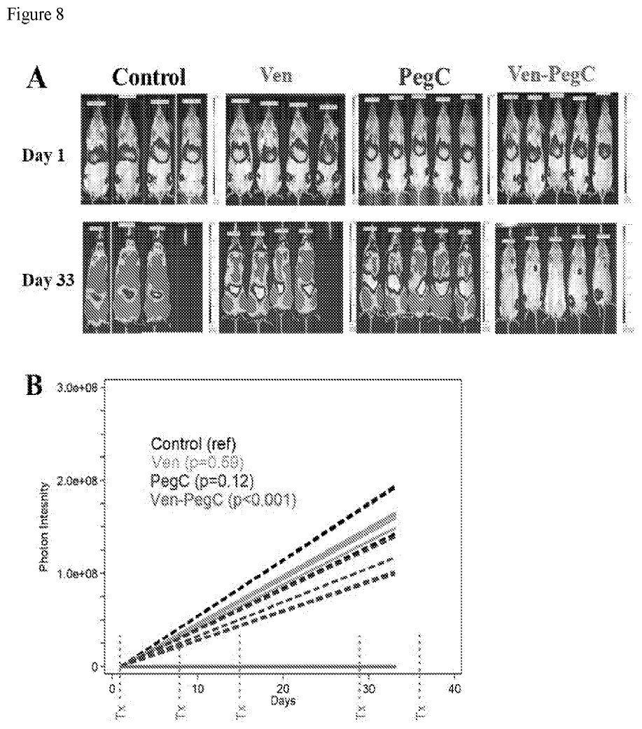

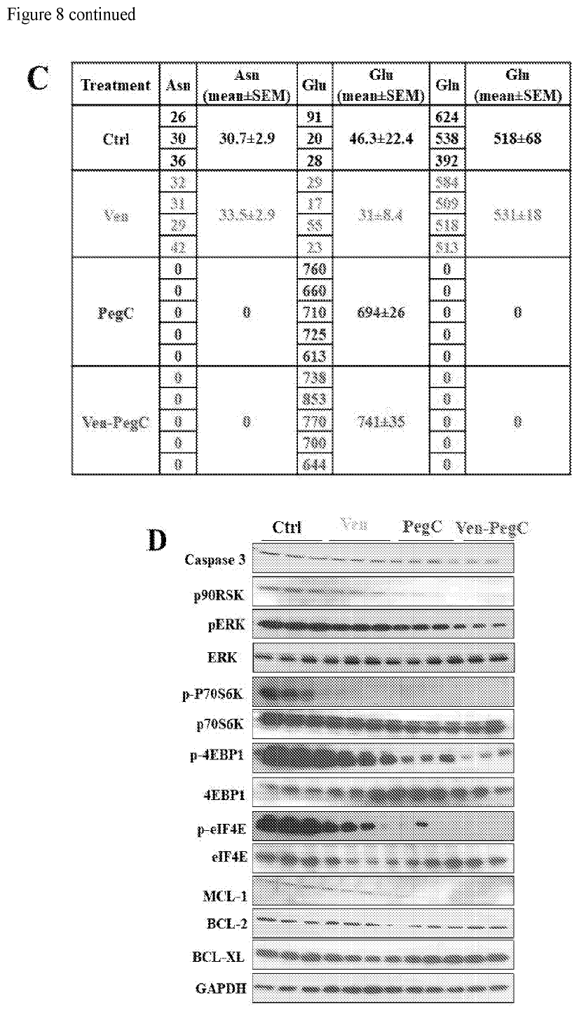

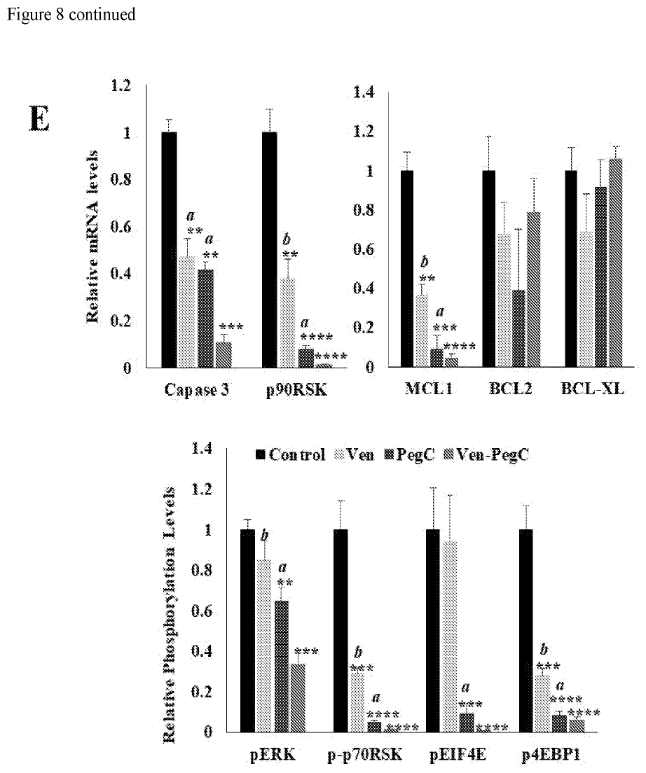

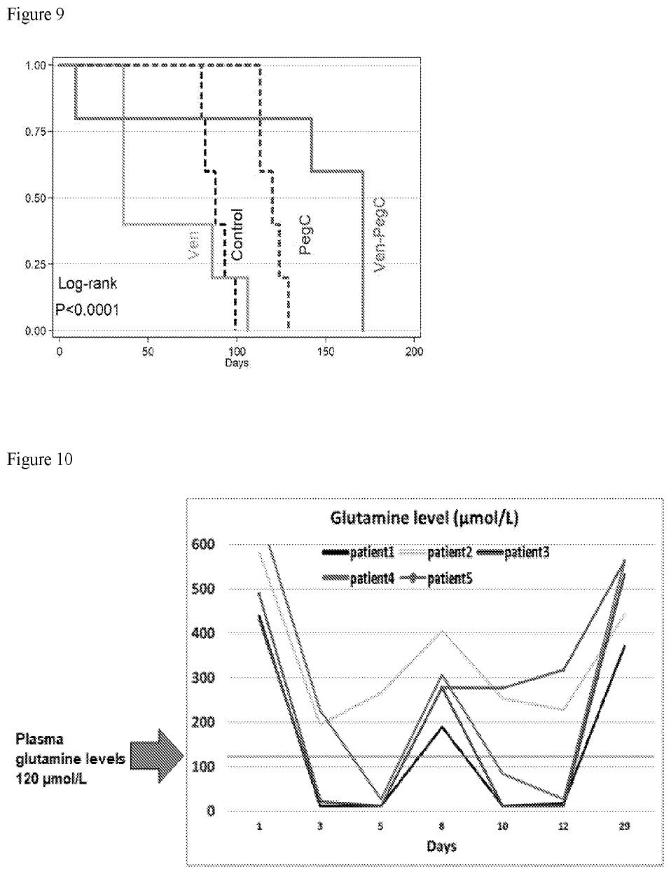

In order to test the novel combination of Ven-PegC in vivo, the experiments presented herein focused on a PDX model of CK-AML, one of the most dismal subtypes of AML. Currently leukemia relapse is the most common cause of treatment failure and mortality for these patients even after stem cell transplantation [39]. This underscores the need for innovative approaches to reduce the relapse rate and improve survival of these patients. In the in vivo efficacy experiment presented herein, survival was estimated and compared by study arm using Kaplan-Meier curves and log-rank tests. Mice died due to leukemia when photon intensity reached a critical level of 1.5×10⁸. Compared with many reported in vivo studies in AML, the study presented herein has two major advantages: 1) mice were treated after confirmation of leukemia engraftment, which resembles real world patient situation as opposed to a prevention model, and 2) the experiments presented herein were carried out for approximately 6 months, which again bears a resemblance to the clinical situation. The experiment was continued for 171 days, during which all 20 mice died. The Kaplan-Meier curves (FIG. 6C) show that all mice in control, Ven, and PegC arms died due to leukemia within 130 days, while none of the mice in the Ven-PegC arm died due to leukemia during the study period (log-rank p-value <0.0001). A sensitivity analysis with outcome as death due to any cause also showed that the Ven-PegC arm was highly superior to the other three arms, with a log-rank p-value <0.0001 (FIG. 9). The tremendous efficacy of Ven-PegC was confirmed in a second cohort of mice with AML45-luc (FIG. 8) as well as in another in vivo model (U937-luc) of human CK-AML (FIG. 7).

In addition to being efficacious, Ven-PegC is also well tolerated. Notably, only one mouse (in the Ven-PegC arm) fell below 80% of the initial weight, and that for only 11 days. All mice gained weight over time. In both immunocompromised and immunocompetent mice, Ven-PegC did not have any negative effect detected on organ function including asparaginase-related adverse events of special interest (i.e. elevated liver or pancreas enzymes, hyperbilirubinemia, coagulation tests and fibrinogen level).

The present invention is thus directed to methods for treating cancer using a combination of an agent that depletes plasma glutamine and an agent that inhibits BCL-2 activity. As discussed in detail herein, it was found for the first time by the inventors that the anti-leukemic drug venetoclax (Ven), in combination with an asparaginase, such pegcrisantaspase (PegC), exhibited strong activity against acute myeloid leukemia (AML), with increased apoptotic activity against AML cell lines in vitro and an enhanced ability to reduce AML tumor burden in vivo. This activity resulted from a coupling PegC-mediated glutamine depletion—resulting in inhibition of synthesis of the extremely short half-lived MCL-1 anti-apoptotic protein—with Ven-mediated antagonism of BCL-2's anti-apoptotic activity.

Combinations of an agent that depletes plasma glutamine and an agent that inhibits BCL-2 activity form the basis of the present invention and they may be used in methods of treating subjects having various forms of cancer and methods of prolonging survival in a subject having cancer.

The invention is generally directed to methods of treating cancer or prolonging survival in a subject by administering therapeutically effective amounts of agents that depletes plasma glutamine and agents that inhibit BCL-2 activity as detailed herein to a subject having cancer. As will be evident from the present disclosure, there are a number of different agents that depletes plasma glutamine that show activity against cancer cells that may be used in these methods. Similarly, there are several different classes of agents that inhibit BCL-2 activity that can be used in the combination therapies. Further, the manner in which the agents are administered to a subject may vary, and include administration of the agents in either order, together in various combinations or separately, sequentially or concurrently, with overlapping or non-overlapping periods of administration. It will thus be clear to the skilled person that the methods of the present invention can be practiced with wide latitude and that the scope of the claims is not narrow.

### Plasma Glutamine-Depleting Agents

Historically, it is thought that the anti-ALL activity of L-asparaginase is a result of the depletion of exogenous amino acid L-asparagine and the failure of malignant cells to generate endogenous L-asparagine. In contrast to ALL cells, asparagine depletion alone is not sufficient for effective cytotoxic activity against AML cells, because glutamine can rescue asparagine-deprived cells in a transamination reaction. It has been shown that AML cells are dependent on glutamine for multiple metabolic purposes including ATP synthesis via mitochondrial glutaminolysis, glutathione synthesis acting as the major cellular antioxidant, glucosamine synthesis, nitrogen donor for nucleotide synthesis, and fatty acid synthesis via cytoplasmic reductive carboxylation. Glutamine starvation and/or interfering with glutamine metabolism can induce apoptosis in AML cells.

The agents that depletes plasma glutamine that may be used in the methods of the present invention include, but are not limited to, E. coli-derived short acting asparaginase, polyethylene glycosylated E. coli-derived asparaginase (pegaspargase and calaspargase pegol-mknl), Erwinia chrysanthemi (recently termed Dickeya dadantii)-derived short acting asparaginase (Erwinaze and recombinant crisantaspase using Pseudomonas fluorescens expression platform), polyethylene glycosylated Erwinia chrysanthemi-derived asparaginase (pegcrisantaspase; PegC). Other examples of agents that depletes plasma glutamine that may be used in the methods of the invention include asparaginase enzymes isolated from other bacteria (e.g. Coliform bacteria, Pseudomonas aeruginosa, Pectobacterium caratovorum, Bacillus subtilis, Serratia marcescens, Staphylococcus capitis), fungi (e.g. Fusarium equiseti, Aspergillus terreus, Aspergillus nieger), actinomycetes (e.g. Streptomyces albidoflavus, Marine Streptomycete strain), and plants (e.g. Soyabean leaves, Ocimum sanctum L, Withania somnifera, Soyabean seeds, Pisum sativum).

### BCL-2 Inhibitory Agents

In the last few years, venetoclax has rapidly become a promising new therapy for AML patients. In November 2018, venetoclax, in combination with azacitidine or decitabine or low-dose cytarabine, was approved by the US Food and Drug Administration (FDA) for the treatment of newly-diagnosed AML in adults who are 75 years or older, or who have comorbidities that preclude use of intensive induction chemotherapy.

The agents that inhibit BCL-2 activity that may be used in the methods of the present invention include, but are not limited to, one or more of venetoclax (Ven), BCL201, and navitoclax.

### Cancer

The methods of the present invention can be used in the treatment of a variety of cancers and in prolonging survival in subjects having a variety of cancers. It will be apparent that particular combinations of agents may differ in effectiveness depending on the type of cancer, the stage and grade of a particular cancer, the physical location of the cancer within the subject, the molecular abnormalities in the cancer, and available means for administering the agents, among other factors.

Exemplary cancers that may be treated via the methods of the invention include cancers overexpressing a BCL-2 family member and cancers overexpressing a kinase or having an increased or constitutively active kinase (e.g., tyrosine kinases, serine/threonine kinases). Examples of cancers exhibiting overexpression of BCL-2 include, but are not limited to, hematologic malignancies (including leukemia, lymphoma, and multiple myeloma) and solid tumors (including prostate, breast, small cell and non-small cell lung cancers, ovarian, neuroblastoma, bladder, colorectal, and head and neck cancers). Examples of cancers overexpressing a kinase or having an increased or constitutively active kinase include, but are not limited to, hematologic malignancies (including leukemia, lymphoma, and multiple myeloma) and solid tumors (including prostate, breast, small cell and non-small cell lung cancers, ovarian, neuroblastoma, bladder, colorectal, mesothelioma, and head and neck cancers).

Exemplary cancers that may be treated via the methods of the invention also include, but are not limited to, leukemias including acute leukemias, such as acute myeloid leukemia (AML; including complex karyotype acute myeloid leukemia (CK-AML)) and acute lymphoblastic leukemia (ALL; including B cell ALL (B-ALL) and T cell ALL (T-ALL)), chronic leukemias, such as chronic myeloid leukemia (CIVIL) and chronic lymphoid leukemia (CLL); lymphoma (including B cell and T cell); myeloma; myelodysplastic syndrome; non-small cell lung cancer; pancreatic cancer; gastric cancer; Kaposi's sarcoma; hepatocellular carcinoma; osteosarcoma; laryngeal squamous cell carcinoma; metastatic uveal melanoma; lung and splenic metastases; advanced non-small cell lung cancer; cervical carcinoma; colorectal cancer; breast cancer; prostate cancer; mesothelioma; and all other hematologic malignancies and solid cancers including brain cancer.

### Pharmaceutical Compositions

The agents used in the methods of the invention may be formulated in pharmaceutical compositions comprising pharmaceutically acceptable carriers, excipients and/or diluents. It will be apparent that depending on the identity of the agents being used, a suitable pharmaceutical composition may comprise a single agent or combinations comprising two or more agents. As used herein, the terms “agent” and “agents” mean the agents that depletes plasma glutamine (e.g. asparaginase) and the agents that inhibit BCL-2 activity (e.g. Ven) as defined herein.

The pharmaceutical compositions may be formulated, for example, for oral, sublingual, intranasal, intraocular, rectal, transdermal, mucosal, pulmonary, topical or parenteral administration. Parenteral modes of administration include without limitation, intradermal, subcutaneous (s.c., s.q., sub-Q, Hypo), intramuscular (i.m.), intravenous (i.v.), intraperitoneal (i.p.), intra-arterial, intramedulary, intracardiac, intra-articular (joint), intrasynovial (joint fluid area), intracranial, intraspinal, and intrathecal (spinal fluids). Any known device useful for parenteral injection or infusion of formulations can be used to effect such administration. In preferred aspects of each of the embodiments on the invention, the pharmaceutical composition is administered to the subject as an intravenous formulation.

Pharmaceutically acceptable carriers, excipients and diluents are those compounds, solutions, substances or materials that can be used to produce formulations of the agents that are suitable to be administered to a subject, such as a human. In particular, carriers, excipients and diluents of the present invention are those useful in preparing a pharmaceutical composition that is generally safe, non-toxic and neither biologically nor otherwise undesirable and that may present pharmacologically favorable profiles, and includes carriers and diluents that are acceptable for veterinary use as well as human pharmaceutical use. Suitable pharmaceutically acceptable carriers, excipients and diluents are well known in art and can be determined by those of skill in the art as the clinical situation warrants. Examples of suitable carriers and diluents include dextrose, water, glycerol, ethanol, propylene glycol, polysorbate 80 (Tween-80™), poly(ethylene)glycol 300 and 400 (PEG 300 and 400), PEGylated castor oil (e.g. Cremophor EL), poloxamer 407 and 188, a cyclodextrin or a cyclodextrin derivative (including HPCD ((2-hydroxypropyl)-cyclodextrin) and (2-hydroxyethyl)-cyclodextrin), hydrophilic and hydrophobic carriers, and combinations thereof. Hydrophobic carriers include, for example, fat emulsions, lipids, PEGylated phospholipids, polymer matrices, biocompatible polymers, lipospheres, vesicles, particles, and liposomes. The terms specifically exclude cell culture medium. More particularly: (1) 5% (w/v) dextrose, or (2) water (e.g., sterile water; Water-For-Injection), may be used as a pharmaceutically acceptable carrier. Pharmaceutically acceptable diluents also include tonicity agents that make the composition compatible with blood. Tonicity agents are particularly desirable in injectable formulations.

Excipients included in a formulation have different purposes depending, for example on the nature of the agent, and the mode of administration. Examples of generally used excipients include, without limitation: stabilizing agents, solubilizing agents and surfactants, buffers, antioxidants and preservatives, tonicity agents, bulking agents, lubricating agents, emulsifiers, suspending or viscosity agents, inert diluents, fillers, disintegrating agents, binding agents, wetting agents, lubricating agents, antibacterials, chelating agents, sweeteners, perfuming agents, flavoring agents, coloring agents, administration aids, and combinations thereof.

The pharmaceutical compositions may contain common carriers and excipients, such as cornstarch or gelatin, lactose, sucrose, microcrystalline cellulose, kaolin, mannitol, dicalcium phosphate, sodium chloride, alginic acid, croscarmellose sodium, and sodium starch glycolate.

The particular carrier, diluent or excipient used will depend upon the means and purpose for which the active ingredient is being applied.

Acceptable methods for preparing the pharmaceutical compositions according to the invention are known to those skilled in the art. For example, pharmaceutical compositions may be prepared following conventional techniques of the pharmaceutical chemist involving steps such as mixing, granulating, and compressing when necessary for tablet forms, or mixing, filling, and dissolving the ingredients as appropriate, to give the desired products for various routes of administration.

### Methods of Treatment

As discussed in the summary of the invention above, methods of the invention include methods of treating cancer in a subject comprising administering therapeutically effective amounts of a first agent that depletes plasma glutamine and a second agent that inhibits BCL-2 activity to a subject having cancer. The first and second agents may be administered in any order, separately or in combination (two agents per combination, or three agents per combination, or four or more agents per combination), sequentially or concurrently, with overlapping or non-overlapping periods of administration. The methods of the invention thus include methods of treating cancer in a subject, comprising concurrently administering therapeutically effective amounts of a first agent that depletes plasma glutamine and a second agent that inhibits BCL-2 activity to a subject having cancer. The methods of the invention thus also include methods of treating cancer in a subject, comprising sequentially administering therapeutically effective amounts of a first agent that depletes plasma glutamine and a second agent that inhibits BCL-2 activity to a subject having cancer.

In a particular aspect, the invention is directed to a method of treating AML in a subject, comprising administering therapeutically effective amounts of an asparaginase and Ven to a subject having AML. The asparaginase and Ven may be administered in either order, alone or in combination, sequentially or concurrently, with overlapping or non-overlapping periods of administration. In one aspect of the method, the asparaginase is PegC.

In a particular aspect, the invention is directed to a method of treating AML in a subject, comprising concurrently administering therapeutically effective amounts of an asparaginase and Ven to a subject having AML. The asparaginase and Ven may be administered together or separately, with partially overlapping or fully overlapping periods of administration. In one aspect of the method, the asparaginase is PegC.

In a particular aspect, the invention is directed to a method of treating AML in a subject, comprising sequentially administering therapeutically effective amounts of an asparaginase and Ven to a subject having AML. The asparaginase and Ven may be administered with partially overlapping or non-overlapping periods of administration. In one aspect of the method, the asparaginase is PegC.

The terms “treating” and “treatment” mean at least the mitigation of cancer, or a disease condition or symptom associated with cancer in a subject that is achieved by a reduction of growth, replication, and/or propagation, or death or destruction of cancer and/or cancer cells, on or in the subject. The terms “treating” and “treatment” include curing, healing, inhibiting, relieving from, improving and/or alleviating, in whole or in part, the cancer or associated disease condition or symptom. The mitigation of cancer or associated disease condition or symptom may be about 100%, 99%, 98%, 97%, 96%, 95%, 90%, 80%, 70%, 60%, 50%, 40%, 30%, 20%, 10%, 5% or 1% in the subject, versus a subject to which the agents taught herein have not been administered. In one aspect, treating means reducing the population of cancer cells causing the cancer in the subject to an undetectable level, where detection is by any conventional means, such as assay a blood sample in the laboratory. In another aspect, treating means complete healing of the cancer, shown by an absence of clinical symptoms associated with the cancer. In a further aspect of the invention, treating means the mitigation of cancer or an associated disease condition or symptom by at least about 90% in the subject. In an additional aspect, treating means the mitigation of cancer or an associated disease condition or symptom by at least about 95% in the subject.

The methods of the invention include also methods of prolonging survival of a subject having cancer comprising administering therapeutically effective amounts of a first agent that depletes plasma glutamine and a second agent that inhibits BCL-2 activity to a subject having cancer. The first and second agents may be administered in any order, separately or in combination (two agents per combination, or three agents per combination, or four or more agents per combination), sequentially or concurrently, with overlapping or non-overlapping periods of administration. The methods of the invention thus include methods of prolonging survival of a subject having cancer, comprising concurrently administering therapeutically effective amounts of a first agent that depletes plasma glutamine and a second agent that inhibits BCL-2 activity to a subject having cancer. The methods of the invention thus also include methods of prolonging survival of a subject having cancer, comprising sequentially administering therapeutically effective amounts of a first agent that depletes plasma glutamine and a second agent that inhibits BCL-2 activity to a subject having cancer.

In a particular aspect, the invention is directed to a method of prolonging survival of a subject having AML, comprising administering therapeutically effective amounts of an asparaginase and Ven to a subject having AML. The asparaginase and Ven may be administered in either order, alone or in combination, sequentially or concurrently, with overlapping or non-overlapping periods of administration. In one aspect of the method, the asparaginase is PegC.

In a particular aspect, the invention is directed to a method of prolonging survival of a subject having AML, comprising concurrently administering therapeutically effective amounts of an asparaginase and Ven to a subject having AML. The asparaginase and Ven may be administered together or separately, with partially overlapping or fully overlapping periods of administration. In one aspect of the method, the asparaginase is PegC.

In a particular aspect, the invention is directed to a method of prolonging survival of a subject having AML, comprising sequentially administering therapeutically effective amounts of an asparaginase and Ven to a subject having AML. The asparaginase and Ven may be administered with partially overlapping or non-overlapping periods of administration. In one aspect of the method, the asparaginase is PegC.

The term “prolonging survival” means extending the life span of a subject having cancer by at least one day versus a subject having the same cancer that does not receive the agents. Prolonged survival includes increasing the life span of the subject by at least: 1, 2, 3, 4 or more weeks, 1, 2, 3, 4, 5, 6, 7, 8, 9, 10, 11, 12 or more months, or 1, 2, 3, 4, 5, or more years.

The amount (dose) of the agents sufficient to have an effect on cancer (additive, synergistic or otherwise) in a subject will vary, for example, in view of the identity of the agents being used in the combination, the physical characteristics of the subject, the severity of the subject's symptoms, the form of the cancer, the identity of the cancer, the formulations and means used to administer the agents, and the method being practiced. The specific dose for a given subject is usually set by the judgment of the attending physician. When the agent is an asparaginase, the amount can be considered in terms of IU and acceptable amounts can range from about 10 to about 50,000 IU/m². As non-limiting, short-acting E. coli asparaginase is typically administered at 6000 IU/m² IM or IV three 3 per week; short-acting Erwinia asparaginase (e.g., crisantaspase) is typically administered at 25,000 IU/m² IM or IV 3 times per week; long-acting pegylated asparaginase (pegaspargase) is typically administered at 2,000 to 2,500 IU/m² IM or IV every 14 days; long-acting pegylated asparaginase (calaspargase pegol-mknl) is typically administered at 2,500 IU/m² IV every 21 days.

When the agent inhibits BCL-2 activity, the amount of the agent is typically between about 10 mg and about 800 mg, and includes doses of between about 100 and about 500 mg. In some aspects, the dose may range from about 10-700 mg, 10-600 mg, 10-500 mg, 10-400 mg, 10-300 mg, 10-200 mg, 10-100 mg, 100-700 mg, 100-600 mg, 100-500 mg, 100-400 mg, 100-300 mg, 100-200 mg, or be about 10, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, or 800 mg.

The timing of administration used in the methods of the invention will vary depending on a number of factors, including whether there is concurrent or sequential administration, the identity of the agents, the identity of the cancer, the physical characteristics of the subject, the severity of the subject's symptoms, and the formulation and the means used to administer the agents, among other factors. However, administration frequencies of the agents will generally include 4, 3, 2 or once daily, every other day, every third day, every fourth day, every fifth day, every sixth day, once weekly, every eight days, every nine days, every ten days, bi-weekly, monthly and bi-monthly, whether the drugs are administered alone or in combination, concurrently or sequentially. In certain aspects, the concurrent or sequential administration is administration once daily. The duration of treatment will be based on the cancer being treated and will be best determined by the attending physician. Under some conditions, treatment will be continued for a number of days, weeks, or months. Under other conditions, complete treatment will be achieved through administering one, two or three doses of the combinations over the entire course of treatment.

The pharmaceutical compositions and the agents of the present invention may be administered via means that include oral, enteral, sublingual, intranasal, intraocular, rectal, intravaginal, transdermal, mucosal, topical or parenteral administration. Parenteral modes of administration include without limitation, intradermal, subcutaneous (s.c., s.q., sub-Q, Hypo), intramuscular (i.m.), intravenous (i.v.), intraperitoneal (i.p.), intra-arterial, intramedullary, intracardiac, intra-articular (joint), intrasynovial (joint fluid area), intracranial, intraspinal, and intrathecal (spinal fluids). Any known device useful for parenteral injection or infusion of agents and formulations can be used to effect such administration. In certain aspects of each of the embodiments of the invention, the agents and pharmaceutical compositions are administered to the subject intravenously.

Depending on the means of administration, the dose may be administered all at once, such as with an oral formulation in a capsule, or slowly over a period of time, such as with an intravenous administration. For slower means of administration, the administering period can be a matter of minutes, such as about 5, 10, 15, 20, 25, 30, 35, 40, 45, 50, 55, 60, 65, 70, 75, 80, 85, 90, 95, 100, 105, 110, 115, 120 or more minutes, or a period of hours, such as about 0.5, 1, 1.5, 2, 2.5, 3, 3.5, 4, 4.5, 5 or more hours. The administration of the dose may be interrupted, such as where the dose is administered via intravenous infusion and the dose is divided into two or more infusion bags. Under such circumstances, the administration of the dose may be interrupted while the infusion bags are changed.

As used herein, the terms “dose”, “unit dose”, “dosage”, “effective dose” and related terms refer to physically discrete units that contain a predetermined quantity of active ingredient or therapeutic drug (agent) calculated to produce a desired therapeutic effect. A single dose is thus a predetermined quantity of an agent of the invention that is administered to a subject.

As used herein, a “subject” is a human, a non-human primate, bird, horse, cow, goat, sheep, a companion animal, such as a dog, cat or rodent, or other mammal.

### Additive and Synergistic Effects

In many instances, the combinations of agents taught herein, i.e., the combination of a first agent that depletes plasma glutamine and a second agent that inhibits BCL-2 activity, target two or more different aspects of a cancer cell, such as two or more different structures, or two or more different pathways, or one or more structures on one hand and one or more pathways on the other. As a result, while the combinations of a first agent that depletes plasma glutamine and a second agent that inhibits BCL-2 activity may have an additive therapeutic effect on a cancer, the combinations may also or alternatively have a synergistic therapeutic effect on the cancer. Synergistic therapeutic effects are those that are substantially greater than what is seen when cancer cells are treated with either drug alone.

### III. EXAMPLES

### Cell Lines and Culturing

The human AML cells, MOLM-14 and MonoMac6 were the kind gift of Dr. Mark Levis from Johns Hopkins University. MV4-11, U937, HL60, THP-1 and K562 cells were purchased from ATCC (Manassas Va.). Primary human leukemia cells were obtained through the institutional (IRB approved) Tumor and Cell procurement Bank at the University of Maryland. Briefly, whole blood was received in sodium ethylenediaminetetraacetic acid (EDTA) tubes, and diluted 1:1 with phosphate buffered saline (PBS). Cells were isolated from diluted whole blood by Ficoll separation in lymphocyte separation medium (Corning Cellgro, Manassas, Va.) spun at 400×g for 30 minutes with no brake. Viable cell numbers were obtained using trypan blue exclusion. All cell lines were grown in 37° C. with 5% CO₂ atmosphere in Roswell Park Memorial Institute (RPMI) 1640 medium (Life technologies, Carlsbad, Calif.) supplemented with heat-inactivated 10% (V/V) fetal bovine serum (FBS) and 1% Glutamax (ThermoFisher Waltham, Mass.). Cell lines were grown and maintained according to ATCC recommendations. For primary cells, cytokines were added including granulocyte-macrophage colony-stimulating factor (GM-CSF, Cell Signaling, Danvers, Mass.) at 5 ng/mL, interleukin 3 (IL3, Cell Signaling, Danvers, Mass.) at 50 ng/mL, and recombinant human thrombopoietin (TPO, Biolegend, San Diego, Calif.) at 25 ng/mL. All cell lines were utilized before 10 passages and treated in exponential growth phase at ˜70% confluency. Human cell line authentication was confirmed using short tandem repeat analysis (STR) from the GenePrint 10 STR typing kit™ (Promega Corp.) (Genomics Core at University of Maryland School of Medicine).

### AML Cells Karyotyping and Mutational Analysis

The assay was performed according to the manufacturer's protocols, except that the GenePrint 5X mouse primer pair mix was added to the reaction to ensure that no mouse DNA contamination was present. Reactions were run on an Applied Biosystems model 3730XL sequencer and a 50 cm array, using GeneMapper software to collect and analyze data. Data was compared to various databases containing STR data for numerous cell lines, including ATCC and Cellosaurus.

FLT3 Fragment Size Analysis was performed for the two common variants in patients with AML: FLT3 internal tandem duplication (ITD) and FLT3 tyrosine kinase domain (TKD) variant at D835. DNA from each cell line was independently amplified by polymerase chain reaction (PCR), using a fluorescently labeled dye attached to the forward primer sequence for each variant. FLT3-ITD was detected as a shift in mobility through a sequencing capillary as detected on an Applied Biosystems model 3730XL. Using GeneMapper software, the size of the ITD and frequency were determined subsequent to PCR amplification. The D835 variant was identified by the ability of the restriction enzyme EcoRV to digest the PCR fragment, as the mutation occurs within an EcoRV restriction site. Using GeneMapper software and running through a sequencing capillary on the Applied Biosystems model 3730XL, the identification of undigested PCR product was indicative of a D835 variant present and the frequency was calculated.

Next-generation DNA sequencing for 31 genes associated with myeloid malignancies (ASXL1, CALR, CBL, CEBPA, CSF3R, DNMT3A, ETNK1, EZH2, FLT3, GATA1, GATA2, IDH1, IDH2, JAK2, KIT, KMT2A, KRAS, MPL, NOTCH1, NPM1, NRAS, PTPN11, RUNX1, SETBP1, SF3B1, SRSF2, TET2, TP53, U2AF 1, WT1, ZRSR2) was performed using Ion Torrent technology. Using the Ion Torrent Ion Chef, protocols developed by the manufacturer were followed for preparing libraries, templating, and chip loading. Loaded chips were transferred to the QuantStudio S5 for sequencing on an Ion Torrent 520 chip, following the manufacturers protocols. After initial data analysis by the S5 software, data was analyzed using Ion Reporter software to identify variants of interest.

### Reagents and Chemotherapeutics

For in vitro studies, PegC was provided by Jazz Pharmaceuticals; Bortezomib, Ixazomib Citrate, Talazoparib and INK-128 were purchased from MedChem Express (Monmouth Junction, N.J.); Carfilzomib, Bafilomycin and venetoclax were purchased from LC Labs (Woburn, Mass.); BPTES, CB-839, Everolimus, Enasidenib (AG-221), hydroxychloroquine were purchased from Selleck Chemicals (Houston, Tex.). All drugs except for PegC were purchased as powder and dissolved in DMSO at 50-100 mM stock solutions and stored at −20° C.

For in vivo studies, PegC was supplied by Jazz Pharmaceutical at 500 IU/ml and stored at 4° C. It was diluted in sterile PBS to the appropriate dosing solution concentration (e.g., 200 IU/kg=20 IU/ml). Ven was dissolved in DMSO at 75 mg/ml, aliquoted and stored at −20° C. It was then formulated fresh on day of dosing as 10% DMSO, 30% PEG400, 60% Phosal PG50. Aza was purchased from Sigma Aldrich and solubilized in sterile saline at 0.05 mg/ml and frozen at −80° C. Aliquots were thawed daily and used immediately.

### IC₅₀ Proliferation Assay

Cell lines and primary cells were seeded into 96-well plates the afternoon prior to treatment. Approximately 18 h later, Ven and Peg-C were serially diluted in vehicle or growth medium and added to cells. Plates were incubated for 72 h for cell lines and 48 h for primary cells prior to addition of water-soluble tetrazolium (WST-1) (Clontech, Mountain View, Calif.). Plates were read after 4 additional hours of incubation at 37° C. using a BioTek Synergy HT plate reader (BioTek, Winooski, Vt.). Data was analyzed and graphed using GraphPad Prism Software (Graphpad, La Jolla, Calif.).

### Cytotoxicity Assay (LDH)

Primary AML cells were plated at a density of 2.5×10⁴ cells/well in 96 well plates in X-Vivo 10 media (Lonza, Walkersville, Md.) then treated the following day with either vehicle (DMSO), Ven (100 μM or 12.5 PegC (0.02 IU/mL or 0.005 IU/mL), or Ven-PegC combination. After 24 h or 48 h, cytotoxicity was measured by lactate dehydrogenase (LDH) release using the Cytotox 96 Non-radioactive Cytotoxicity Assay (Promega, Madison, Wis.). Percent cytotoxicity was calculated as (experimental release/maximum release)×100.

### Cell Survival Assay

MOLM-14, MonoMac6 and primary cells were seeded onto 96 well plates and treated with Ven and/or PegC as previously described. Cells were incubated for 48-72 h then counted using Trypan blue exclusion on the Countess automated cell counter (Life Technologies, Carlsbad, Calif.). Cell counts were performed in duplicates, and the averages were graphed using Graphpad Prism software.

### Potentiation and Synergism Assays

The effect of PegC to potentiate the cytotoxicity of Ven was investigated by conducting the proliferation assay with IC₅₀ of Ven in the presence of IC₁₀, IC₂₀ and IC₃₀ of PegC. Agents were added simultaneously and exposed for 72 h, then assays were terminated with WST-1. IC_{50S} for the combined agents were calculated by GraphPad Prism. For synergism, both agents were added in fixed ratios (e.g., ¼×IC₅₀, ½×IC₅₀, 1×IC₅₀, 2×IC₅₀, 4×IC₅₀) for 72 h and assays terminated with WST-1. The optical density units from the WST-1 assay were analyzed by median effect analysis using Combosyn software (free online software based on the Chou Talalay thereom) [18]. Combination Indices (CI) are generated; CI<1 synergistic, CI=1 additive, CI>1 antagonistic.

### Cell Cycle Analysis

MOLM-14 cells were plated overnight then treated with either vehicle, Ven (5.2 μM), PegC (0.025 IU/mL), or Ven-PegC combination. At 48 h, cells were fixed and permeabilized in ice-cold 70% ethanol for 2 h at −20° C. then washed with cold PBS. Cells were resuspended in staining buffer (PBS with 0.5% BSA and 2 mM EDTA) with RNAase (100 μg/mL, Sigma) and propidium iodide (BioLegend, San Diego, Calif.) and incubated for at least 1 h at 4° C. Samples were run on the BD FACS Canto II and analyzed using FCS Express V6 (De Novo Software).

### Western Blotting Analysis

Cells were lysed with radioimmunoprecipitation assay (RIPA) buffer (Thermo Fisher Scientific) supplemented with cOmplete™, EDTA-free Protease Inhibitor Cocktail (Sigma Aldrich). After thorough mixing and incubation at 4° C. for 30 min, lysates were centrifuged at 10,000 g at 4° C. for 10 min, and supernatants collected. Protein content of lysates was determined, and lysates separated by 4-12% polyacrylamide gel electrophoresis (SDS-PAGE), and electro-transferred onto polyvinylidene difluoride (PVDF) membranes. After blocking with 5% non-fat milk in tris-buffered saline, 0.1% Tween 20 (TBST), membranes were incubated with primary antibodies at 4° C. overnight, followed by 1:3000 horseradish peroxidase (HRP)-conjugated secondary antibody (Santacruz Biotechnology, Dallas, Tex.) for 1 h. Bands were visualized using Pierce Enhanced Chemiluminescence (ECL) Western Blotting Substrate (Thermo Fisher Scientific, Waltham, Mass.). Densitometry analyses were performed using Image Studio (Licor Biosciences) and presented as ratio of target band signal intensity to Actin band signal intensity.

Analysis of m⁷GTP-Sepharose-Bound Proteins

The affinity purification of proteins associated with the m⁷GTP Sepharose (Jena Biosciences, Germany) was performed similarly to that described earlier [31]. Cell lysates were prepared after 24 h of treatment and incubated with m⁷GTP-sepharose for 2 h in cap-binding buffer (40 mM 4-(2-hydroxyethyl)-1-piperazineethanesulfonic acid [HEPES], pH 7.6; 120 mM NaCl; 1 mM EDTA; 0.3% 3-[(3-cholamidopropyl)dimethylammonio]-1-propanesulfonate. [CHAPS]). The beads were washed at room temperature three times in cap-binding buffer and boiled with 2× loading dye followed by separation on 4-12% SDS-PAGE and probed for specific antibodies.

### RT-PCR

Total RNA was extracted using Tri Reagent (Sigma Aldrich) following manufacture's protocol. 2 μg of total RNA was treated with DNase I (NEB) and reverse transcribed into cDNA using High-Capacity cDNA Reverse Transcription Kit (Thermo Fisher), followed by qPCR with Power SYBR green PCR master mix (Thermo Fisher) on QuantStudio 7 Flex Real-Time PCR System (Thermo Fisher).

### Polysomal Fractionation

Polysomal fractionation was performed as reported earlier [31]. Briefly 100 million cells were lysed in polysomal lysis buffer and fractionated using a linear sucrose gradient (10-50%). RNA was isolated and cDNA was prepared as mentioned earlier. RNA isolated from polysomal fractions were mixed in equal ratios and was utilized for RNA-Seq.

### Transcriptome and Translatome Bioinformatic Analyses

The transcriptome and translatome samples were sequenced at the Institute for Genome Sciences (IGS), Baltimore, Md. using the Illumina HiSeq sequencing platform. Raw sequencing reads generated for each sample were analyzed using the CAVERN analysis pipeline [52]. Read quality was assessed using the FastQC toolkit [53] to ensure good quality reads for downstream analyses. Reads were first aligned to the human reference genome build GRCh38 using HISAT2, a splice-aware alignment software for mapping next-generation sequencing reads [54]. Reads were aligned using default parameters and the strand-specific protocol to generate the alignment BAM files. Read alignments were assessed to compute gene expression counts for each gene using the HTSeq count tool [55] and the human reference annotation (GRCh38.91). The raw read counts were normalized for library size and utilized to assess differential gene expression between the control and drug-treated groups using the R package ‘DESeq’ [56]. P-values were generated using a modified Fisher's exact test implemented in DESeq and then corrected for multiple hypothesis testing using the Benjamini-Hochberg correction method. Significant differentially expressed genes between conditions were determined using a false discovery rate (FDR) of 5% and a minimum fold-change of 2×.

### In Vivo Tolerability and Safety

For all studies, mice were housed under pathogen-free conditions at University of Maryland Baltimore Association for Assessment and Accreditation of Laboratory Animal Care (AAALAC) accredited facility. All experiments were conducted in compliance with Public Health Service (PHS) guidelines for animal research and approved by UMB Institutional Animal Care and Use Committee. NRG (NOD.Cg-Rag^{tm1Mom}IL2rg^{tm1Wjl}) mice were purchased from Jackson Labs (Maine) and bred by University of Maryland Veterinary Resources. Initially mice (3 mice/group) were dosed with a very high dose (1000 and 500 IU/kg) of PegC (IV) monotherapy weekly ×2 weeks. Due to significant weight loss, 250 IU/kg was chose as MTD.

To test the MTD of the combination of Ven and PegC, female NRG mice (3 mice/group) were dosed with 100 mg/kg Ven (purchased from LC Labs and formulated in 10% DMSO, 30% PEG400, 60% Phosal PG50) orally via gavage 5 days per week, and 250 IU/kg PegC (kind of gift of Jazz Pharmaceutical, formulated in PBS) and their combination. Mice were weighed and monitored 5 days per week.

To test the effect of Ven-PegC on the laboratory parameters, CD1 mice were purchased from Envigo (Frederick, Md.). Female and male mice were dosed either with vehicle (Control) or Ven (75 mg/kg, PO 5 days per week) and/or PegC (200 IU/kg, IV weekly). Mice were euthanized 15 days later and exsanguinated one hour after last dose. Whole blood and plasma was sent to VRL laboratory (Gaithersburg, Md.) for complete blood count (CBC) and clinical chemistry analysis. A similar experiment was conducted with combination of Ven and pegaspargase.

### Establishment of AML45-luc-FYP Model

Primary patient cells (AML45) were obtained by the Civin Lab from University of Pennsylvania (kind gift of Drs. Martin Carroll and Alexander Perl) under an Institutional Review Board-approved research protocol. AML45 was seeded at 1×10⁶ cells in 500 μL RPMI plus 10% FBS supplemented with 500 nM StemRegenin 1 (SR1, StemCell Technologies, Vancouver, Canada) and 8 mg/mL of polybrene in a 24 well plate. Murine Stem Cell Virus (MSCV)-derived promoter driving luc2 internal ribosome entry site (IRES) yellow fluorescent protein (YFP) lentivirus (kind gift of Dr. Sharyn Baker of The Ohio State University and Viral Vector Core of St. Jude University) was added to the well at a MOI of 25-30. After 48 h, cells were injected IV via lateral tail vain into NSG/NRG mice. Bone marrow and spleens were collected from mice after 42 days and YFP⁺/huCD33⁺ (Cat. #551378, BD, Franklin Lakes, N.J.) cells were sorted and collected using Fluorescence-activated cell sorting (FACS) Aria II. The transduction/transplantation technique was repeated 3 times until the huCD33⁺ AML45 cells had a YFP⁺ mean fluorescent intensity >10⁶.

Transduced cells were injected IV into NSG (NOD.Cg-Prkdc^{scid}IL2rg^{tm1Wjil}/SzJ) or NRG mice. Mice were imaged on the Xenogen IVIS Spectrum at the Imaging Core of the University of Maryland School of Medicine and after approximately 100 days, luminescent AML cells were detected. To image, mice were injected with 150 mg/kg luciferin (Perkin Elmer, Hopkinton, Mass.) via intraperitoneal (IP) injection. At approximately four months, mice were euthanized, bone marrow extracted and cells were serially re-transplanted by aseptically flushing bone marrow cells from femurs and tibia. Bone marrow aspirates were filtered through a 100 μm filter, filtrate was centrifuged at 300 g for 10 min, counted by Trypan blue and reinjected into recipient mice. At the penultimate passage, bone marrow cells were extracted and then aseptically sorted for YFP BD FACS Aria II platform at UMGCCC's flow cytometry core. A pure population of AML45-luc-YFP cells were viably frozen and injected into recipient mice.

### In Vivo Efficacy in AML45-luc-YFP

Viably frozen AML-45-YFP-luc cells were thawed, washed with PBS and injected into three NSG mice and allowed to expand in vivo. After 4 weeks, mice were euthanized, cells isolated from bone marrow aseptically and lx10⁶ AML45-luc-YFP cells were injected IV into NRG mice for the experiment. Three to ten days later, mice were imaged and robust AML engraftment was confirmed. Mice were sorted into four groups of five mice with the equal mean leukemia burden among the groups. Dosing began on day of sorting with vehicle, Ven (PO, 75 mg/kg, 5 days per week), PegC (IV, 200 IU/kg, once weekly), or Ven-PegC. Mice were imaged weekly by Xenogen to monitor the photon intensity as the surrogate for leukemia burden. Mean photon intensity was calculated by averaging the maximal photon intensity for each mouse on each day of imaging.

In Vivo Efficacy in U937-luc

U937 cells were cultured in RPMI 1640 supplemented with 10% FBS and 1× Glutamax, according to ATCC guidelines. On the day of transduction, actively growing cells were counted and seeded into a 24-well plate at 10⁵ cells per 500 μl. Cells were transduced for a period of 72 h with YFP-Luciferase lentiviral supernatant in the presence of 8 μg/ml polybrene. Cells were then harvested, washed once with PBS, and plated into T25 cell culture flasks for propagation. Actively growing, transduced cells were subjected to sterile sorting using the BD FACS Aria II platform (BD Biosciences, San Jose, Calif.). U937-luc cells (0.25×10⁶) were injected IV into NRG mice. Three days post cell injection, mice were imaged and robust engraftments were confirmed. Mice were sorted into 6 groups (Control, Ven, PegC, Ven-PegC, Aza, and Aza-Ven) of 5 mice with the equal mean leukemia burden among the groups. Treatment started on day of sorting.

### Pharmacodynamic Assay

AML45-luc model was generated as described above. Mice were dosed with vehicle, Ven, PegC or their combination (FIG. 8 legend). On day 39 post start of dosing, mice were euthanized. Plasma was isolated from whole blood after exsanguination and delivered to the Biochemical Genetics lab (University of Maryland Baltimore) on ice. Bone marrow cells was extracted from both femurs as described above. Cells were pelleted and lysed in RIPA buffer (above) and used for western blot analysis.

### Study Design

The primary objective of the animal studies was to determine the efficacy of Ven-PegC combination in reducing leukemia burden in mice engrafted with human AML cells with complex karyotype isolated directly from a patient (AML45-luc) or with an AML cell line with complex karyotype (U937). The secondary objectives were to determine the safety and tolerability of Ven-PegC as measured by weight change in mice over time, and blood tests including complete blood count (CBC), and comprehensive panel (CMP) including pancreatic enzymes and coagulation markers. The exploratory pharmacodynamic objectives of the animal study were to measure the effect of PegC with or without Ven on the plasma Glutamine, Asparagine and Glutamate levels as well as effect of Ven-PegC on the expression of relevant proteins related to cellular ribosomal protein synthesis including p90RSK, p70S6K, 4EBP1, and eIF4E and related to resistance to Ven including MCL-1, BCL-2 and BCL-XL. It was hypothesized that Ven-PegC therapy would be superior compared with each agent alone and vehicle for treating poor risk AML.

AML45 cells were derived from a patient with history of MDS who progressed to AML and later relapsed with a complex karyotype, 46,X,del(X)(p11.4),t(1;3)(p36.1;p25), t(2;?13)(q21;q13),t(2;11)(q12;q25),add(3)(p22),der(3)t(3;?;8)(p22;?;q12.1),der(5)del(5)(p14)t(2; ?;5)(q11.2;?;q35),?t(8;16)(p11.2;p13.3),der(15)t(3;?;8)(q24;?;q12.1),t(17;22)(q21;q13)). AML45 was transduced with lentivirus to express luciferase and yellow fluorescent protein (YFP) (AML45-luc). AML45-luc (1×10⁶ cells) were injected IV into NRG mice, and after confirmation of engraftment, mice were randomly assigned to be treated with vehicle, Ven, PegC, and Ven-PegC. Mice were imaged weekly and survival was monitored. Ven was dosed at 75 mg/Kg once daily on days 1-5, 8-12, 23-24, 30-33, 78-82, 86-89, 99-103. PegC was dosed at 200 IU/Kg weekly on days 1, 8, 23, 30, 78, 87, 99. No treatment was administrated between Week 5 and Week 11. One mouse in the Ven-PegC group died in the second week due to a technical error. Leukemia burden was measured by photon intensity.

To confirm the observed outstanding anti-AML activity of Ven-PegC, this combination was tested in another xenograft complex karyotype human AML model. Efficacy of single agents Ven, PegC, azacitidine (Aza), and combination of Aza-Ven and Ven-PegC were tested in U937-luc. Of note, Aza and Aza-Ven were selected as comparator arms since this combination is commonly used as a current standard-of-care regimen for treatment of patients with complex karyotype AML. U937-luc (0.25×10⁶) were injected IV in NRG mice. Following engraftment, treatment was initiated: PegC (200 IU/kg IV weekly) and/or Ven (75 mg/kg PO 5 days weekly) and/or azacitidine (0.5 mg/kg subcutaneous 5 days weekly). Mice were imaged weekly and survival monitored. Ven was dosed on days 1-5, 8-10. PegC was dosed on days 1, 8. Aza was dosed on days 2-5, 8-12. In the Ven-PegC group (the only mice to live beyond day 18), mice were treated with one more dose of PegC and 3 more doses of Ven. Leukemia burden was measured by photon intensity.

For secondary objectives, to determine single agent tolerability of PegC, NRG mice (3 mice/group) were dosed IV with 1000 IU/kg and 500 IU/kg once weekly for two weeks. To test tolerability of Ven-PegC combination, NRG mice (3 mice/group) were dosed with 100 mg/kg Ven PO 5 days per week and 250 IU/kg PegC. Mice were weighed and monitored 5 days per week. For evaluation of laboratory adverse events of interest, CD1 mice were dosed either with vehicle (Control) or 75 mg/kg PO 5 days per week Ven and/or 200 IU/kg PegC IV weekly. Fifteen days after start of dosing, mice were euthanized and exsanguinated one hour post last dose. Whole blood and plasma were analyzed for CBC and CMP including transaminases, bilirubin, pancreatic enzymes and coagulation markers (fibrinogen and PT/PTT).

For exploratory objectives, after confirmation of engraftment of AML45-luc, mice were treated with Ven (days 1-5, 8-12, 15-19, 29-33 and 36-38), PegC (days 1, 8, 15, 29 and 36) and Ven-PegC at doses similar to the efficacy study. Mice were imaged on day 33 and euthanized on day 39. Plasma was isolated and used for amino acid analysis, an important pharmacodynamic endpoint. Bone marrow cells were harvested and analyzed for the expression of proteins of interest.

### Statistical Analysis

For IC_{50S} in the AML cell lines, data are presented as means±standard deviations (SD) and p<0.05 was considered as significant. For the in vitro mechanistic studies, data presented as means±standard error of means (SEM) with p<0.05 as significant. Analysis of variance (ANOVA) was used to compare differences among multiple groups followed by Bonferroni's post hoc correction. Statistical parameters including sample size and statistical significance are reported in the figures and corresponding figure legends. In vivo sample size was determined based on achieving 80% power with a type I error rate of 5% and an anticipated difference of ˜20% in mean leukemia burden between arms. All available data points were included in the final analysis.

Survival of mice with engrafted AML cells was estimated using Kaplan-Meier estimators and compared across the study arms using log-rank tests. All analyses were performed by comparing death due to leukemia, i.e., only if photon intensity reached 1.5×10⁸(primary analysis). Death due to reasons other than leukemia such as human error were censored at the date of death. Line graphs of photon intensity were drawn for all mice with engrafted leukemia cells, during 171 days of follow-up, and were compared across the study arms using linear random effects models. Similarly, weights were charted for each of the mice, as a percentage of their initial weight, versus follow-up date.

The distribution of 39 amino acids was compared between 10 mice that did and 7 mice that did not receive PegC using Wilcoxon rank sum (Mann-Whitney U) tests.

Statistical analyses were performed using Stata 14.2, GraphPad Prism, Image Studio and R statistical package. Two-sided p-values <0.05 and 95% confidence intervals that did not include 1 were considered as statistically significant.

### Example 1—Anti-AML Activity of Ven is Significantly Potentiated by PegC In Vitro

Exposure to PegC (alone) decreased in vitro proliferation of all 7 tested human AML cell lines (HL-60, K562, MOLM-14, MonoMac-6, MV4-11, THP-1, U937)) in a concentration-dependent manner (data not shown). IC_{50S} of PegC in the AML cell lines ranged from 0.0001 to 0.049 international unit per milliliter (IU/mL), indicating potent single-agent activity even when compared with ALL cell lines [16]. Importantly, these concentrations are pharmacologically relevant and can be achieved in patients [17].

The anti-AML activity of 12 other antineoplastic drugs was tested that, at least in theory, could be related mechanistically to PegC, including inhibitors of BCL-2, glutaminase, autophagy, proteasome, and mammalian target of rapamycin (mTOR). Proteasome inhibitors had potent in vitro activity against the AML cell panel (data not shown); co-exposure to PegC plus proteasome inhibitors demonstrated no synergy in any AML cell line tested (data not shown).

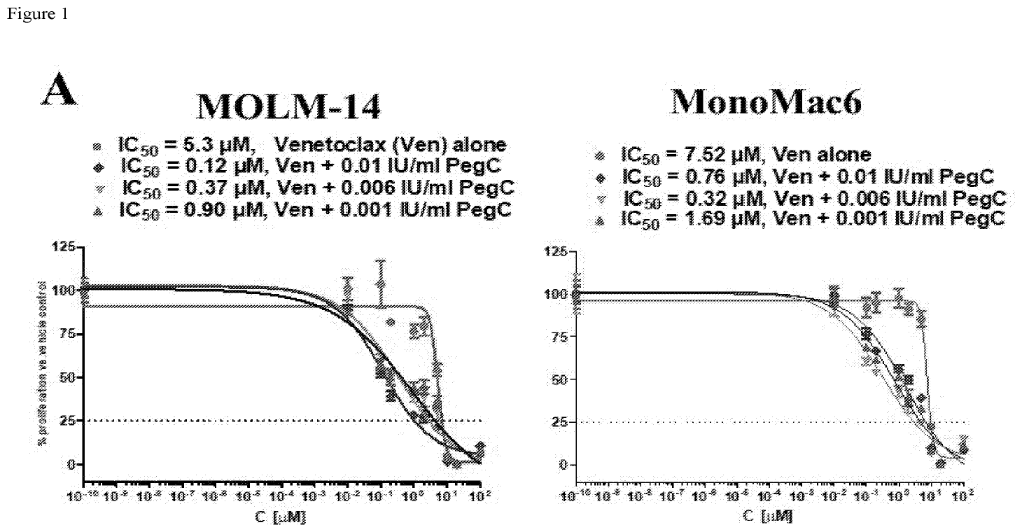

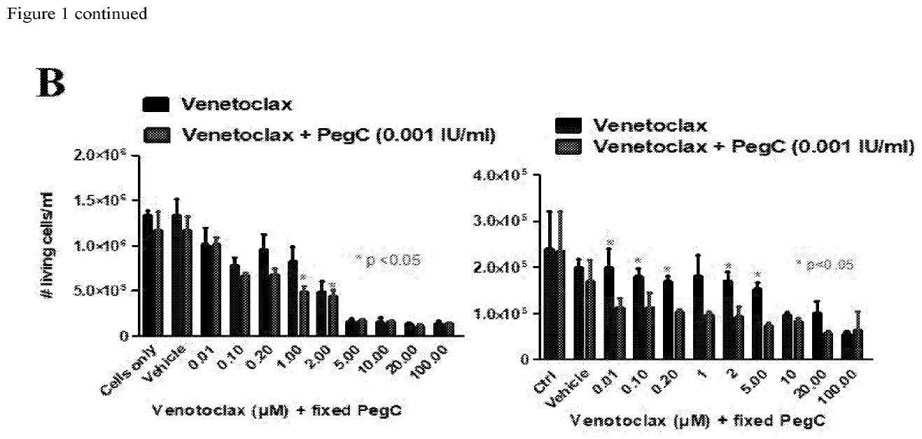

Ven has micromolar activity against 6 of 7 AML cell lines studied, and Ven has clinical efficacy against AML, especially in combination with other agents. Co-exposure to a low concentration of PegC (0.01 IU/mL) diminished the IC₅₀ of Ven by ˜50-fold to 0.12 μM in MOLM-14 and by ˜10-fold to 0.76 μM in MonoMac6 cells (FIG. 1A). Trypan blue exclusion assays confirmed these results in both the MOLM-14 and MonoMac6 AML cell lines (FIG. 1B). The Ven-PegC combination synergistically inhibited leukemia growth (combination index [CI]<1), as determined by the Chou-Talalay method [18].

The anti-AML activity of PegC, Ven and the Ven-PegC combination was confirmed against primary AML cells from two patients (AML29 and AML31). Single-agent PegC and Ven decreased proliferation of AML29 and AML31 in vitro (data not shown). Potentiation of AML cell killing was also observed in AML29 and AML31 (FIG. 1C). Cell cycle analysis showed Ven-PegC combination induced cell cycle arrest in MOLM-14 cells, as evident by the reduction of cells in the S and G2 phases (FIG. 1D).

To evaluate whether the observed promising potentiation and synergism between PegC and Ven is unique, other combinations were tested with PegC or Ven. Addition of PegC to proteasome inhibitors bortezomib and carfilzomib did not cause potentiation in any AML cell line tested (data not shown). Addition of Ven to decitabine or azacitidine (the clinically approved and widely used regimens) showed modest additive effect (data not shown). These results suggest that the potentiation effect appears to be specific for the Ven and PegC combination.

Enhanced Ven-PegC-mediated apoptosis was observed versus each agent alone, as demonstrated by decreased caspase 3 in two AML cell lines (FIG. 1E). To investigate whether this increased apoptosis is due to downregulation of anti-apoptotic proteins, the expression of anti-apoptotic proteins BCL-2 and BCL-XL in MOLM-14 cell lines after treatment with Ven and/or PegC was measured. Neither Ven alone nor Ven-PegC altered intracellular levels of BCL-2 or BCL-XL proteins (data not shown), suggesting that other mechanism(s) explain their synergistic cytotoxicity.

### Example 2—Ven-PegC Alters Transcription and Robustly Inhibits p90RSK Transcript in AML Cells

Whole-transcriptome/gene expression profiling (GEP) was first performed in MOLM-14 cells treated with Ven, PegC or Ven-PegC using RNA sequencing (RNA-seq). Principal Component Analysis (PCA) of this transcriptomic data indicated highly correlated replicates in each treatment group and diverse clusters among different conditions (i.e. vehicle control, Ven, PegC, and Ven-PegC) on the basis of their GEP (FIG. 2A). The RNA-Seq analysis identified more than 30,000 genes in each dataset with relatively high expression levels, covering more than 92% of exonic regions. Using multivariable linear regression analysis, significant gene expression changes were observed following Ven (n=277) and PegC (n=71) monotherapy, as compared to vehicle control, as shown in the Heatmap derived from transcriptome data analysis (FIGS. 2B, 2C). A robust modification of mRNA levels (n=677) was noted after treatment with the Ven-PegC combination, as compared to vehicle (FIG. 2C). Treatment of MOLM-14 cells with Ven-PegC combination induced changes in the RNA transcripts of 677 genes (282 upregulated/395 downregulated), which was in clear contrast to the number of RNA transcripts modified with each individual agent alone (FIG. 2C). These results corroborate the in vitro findings that Ven-PegC combination acts synergistically in AML cells.

There was only a low correlation of GEP for PegC vs Ven (R=0.58, data not shown) and PegC vs Ven-PegC (R=0.49, data not shown). However, there was a high correlation of GEP for Ven vs Ven-PegC (R=0.85, FIG. 2B). Treatment with single-agent Ven or PegC resulted in alteration of expression of different gene; e.g. compared to control, PegC significantly upregulated its signature gene asparagine synthetase (ASNS), as expected (data not shown) [19]. It also increased expression of the stress-induced GPI-anchored gene ULBP1 as well as AMD2, an enzyme prominent in polyamine biosynthesis (data not shown). In contrast, Ven significantly downregulated the glycolytic gene hexokinase 3 (HK3) and the signal transduction phosphatase gene DUSP4 (data not shown). Additionally, Ven and PegC changed the expression of different solute carrier transporter genes (data not shown).

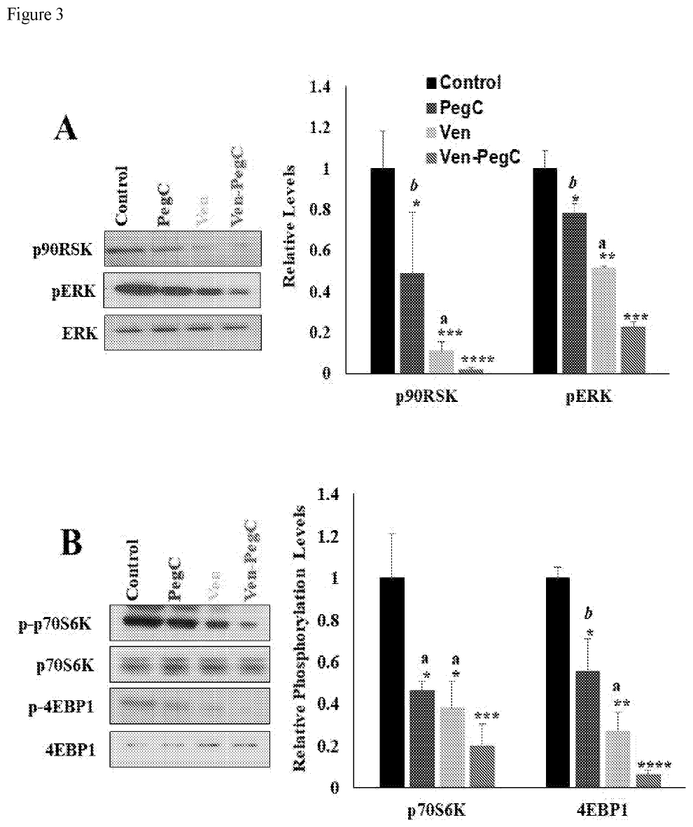

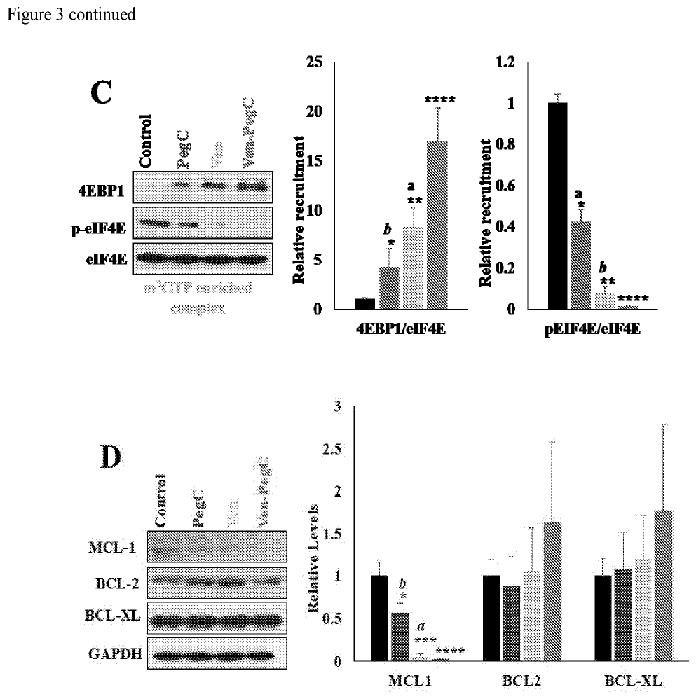

Next, attention was turned to candidate gene(s) that were modulated by the Ven-PegC combination treatment. 23 genes were identified that were modulated by all 3 drug treatment groups: Ven, PegC and Ven-PegC (FIG. 2D). qRT-PCR of selected genes modified by Ven-PegC, compared with the control gene IL17R, confirmed transcriptome analysis results (data not shown). Among these 23 genes, it was observed that p90 ribosomal S6 kinase 2 (p90RSK2) levels were significantly decreased in cells treated with PegC or Ven (p<0.005) and robustly decreased with Ven-PegC (p<0.001) (FIG. 2E). Western blot analysis showed significant decreases in p90RSK protein levels in MOLM-14 cells treated with PegC or Ven, and enhanced reduction with Ven-PegC (FIG. 3A).

p90RSK is known to play an important role in the Ras-mitogen-activated protein kinase (MAPK) signaling cascade. Activated Ras-extracellular signal-regulated kinase (ERK1/2) directly phosphorylates and activates p90RSK, which, in turn, activates multiple signaling events associated with cell proliferation and survival [20], such as translocation of mRNA to polyribosomes and new protein translation [21]. Activation of ERK is reported to regulate BCL-2 expression [22-24], which prompted study of the effect of PegC and/or Ven on ERK signaling, a common mediator of chemoresistance in AML [25]. Upon treatment of MOLM-14 and MonoMac6 cells with Ven or PegC, phosphorylation of ERK was markedly reduced; more robust reduction was noted upon Ven-PegC exposure (FIG. 3A). Diminished ERK signaling and decreased p90RSK expression may hamper cell growth through interference with mRNA translation [26].

### Example 3—Ven-PegC Enhances eIF4E-4EBP1 Interaction on Cap-Binding Complex, Inhibits Cap-Dependent Translation and Directly Impacts the mTOR Pathway

Resistance to BCL-2 inhibitors such as Ven has been linked to activation of the AKT pathway as well as upregulation of MCL-1 [27,28]. Indeed, treatment of non-Hodgkin lymphoma cell lines with PI3K, AKT and mTOR inhibitors overcame resistance to Ven by reducing MCL-1 [27]. Since asparaginase-induced glutamine and asparagine depletion was reported to hinder mTOR signaling in ALL cell lines [29], it was reasoned that co-treatment with PegC and Ven would inhibit mTOR signaling. As reported in ALL cells [29], a significant decrease in phosphorylation of p70S6K (p-p70S6K) as well as its substrate 4EBP1 (p-4EBP1) was noted upon treatment with Ven-PegC (FIG. 3B), significantly more than for each individual drug (FIG. 3B). Similar results were observed in MonoMac6 cells (data not shown).

Since these decreases in phosphorylation of mTOR substrates and in p90RSK protein levels suggested that Ven-PegC may suppress protein translation, m⁷GTP enrichment experiments were performed and recruitment levels of 4EBP1 and pSer209-eIF4E (the active form of eIF4E) were probed on the cap-binding complex. A sequential increase was observed in 4EBP1 recruitment cap complexes (Ven-PegC>Ven>PegC) (FIG. 3C). In contrast, a significant reduction in the phosphorylation of eIF4E was noted on the cap complex (FIG. 3C).

Since the MCL-1 protein has an extremely short half-life, cellular MCL-1 protein expression is highly dependent on 4EBP1/eIF4E activity [30,31]. A significant decrease was observed in MCL-1 protein levels in AML cells treated with Ven-PegC (FIG. 3D), whereas BCL-2 and BCL-XL protein levels were not affected (FIG. 3D).

### Example 4—Ribosomal Profiling Shows Minimal Translational Complex Formation Upon Ven-PegC Treatment

These results indicated a strong tethering of 4EBP1 with eIF4E in formation of inactive translational initiation complexes, suggesting a global reduction by Ven-PegC in cap-dependent protein translation which prompted an examination of ribosomal profiling as measured by the formation of actively translating ribosomes. After treatment of MOLM-14 cells with PegC and/or Ven, ribosomal fractions were enriched by sucrose gradient followed by RNA isolation and sequencing. The area under the translation initiation complex (80S) decreased with all drug treatments, as compared to vehicle (data not shown). Polysomal capacity decreased significantly with PegC treatment, compared to Ven and control treatment, while very minimal translational complex formation was observed with Ven-PegC treatment, indicating a robust reduction of protein translation (data not shown). To assess the in-depth impact of pharmacological treatment on overall translation of the open reading frames (ORFs), the RNA-Seq of the polysomal enriched fraction (data not shown) was performed. ˜90 million reads mapping to exonic regions of the genome were identified, resulting in more than 17,000 genes with transcript per million mapped reads (RPKM) values >0.1 (data not shown). Among the differentially-bound genes (DBGs) with control versus drug treatments detected in this experiment (FIG. 4A), there was a global positive correlation between the expression noted in the Ven and Ven-PegC groups, as compared with Ven and PegC or PegC and Ven-PegC (data not shown), an observation consistent with the transcriptome profile.

Next, to assess overall mRNA translation efficiency (TE), the reads per kilobase of RPKM measured by polysomal sequencing over the RPKM measured by RNA sequencing of transcriptome (RNA-seq) was log 2 divided, and then changes in TE induced with drug treatments were determined. Owing to the stochastic nature of massively parallel sequencing, the reliance over quantification of gene expression is dependent on its sequencing depth. Therefore, the estimated expression levels of weakly expressed genes will have greater variability than highly expressed genes. To mitigate this effect, the uniformly low abundance detected genes were filtered to improve the detection of true differential expression using the model described by Bourgon et al. [32]. It should also be noted that there was minimal translation was noted upon treatment with Ven-PegC, resulting in very low abundance of transcripts. A ratio above 0 (log 2-transformed) in the high TE group indicates a more sensitive reporting of translation for ribosomal profiling, while a ratio below 0 in the low TE group also indicates superior sensitivity for the ribosomal profiling approach (FIG. 4B). Translation of 130, 234 and 93 mRNA transcripts increased after PegC, Ven and Ven-PegC treatments, respectively, while translation of only 86, 15 and zero mRNA transcripts decreased (FIG. 4C). FIG. 4D represents the overlap between target genes altered by treatments. To minimize the false discovery rate, these results were validated by qPCR on the isolated RNA from the sucrose gradient-separated cell lysates and compared their relative expression with RNA-Seq for individual genes (data not shown). As a control, the overall mRNA expression of glyceraldehyde 3-phosphate dehydrogenase (GAPDH) was unaffected by any treatment (data not shown). Together these results demonstrate genome-wide dysregulation of translation upon treatment with Ven-PegC as a mechanism of cytotoxicity in AML cells.

### Example 5—Ven-PegC is Well Tolerated In Vivo

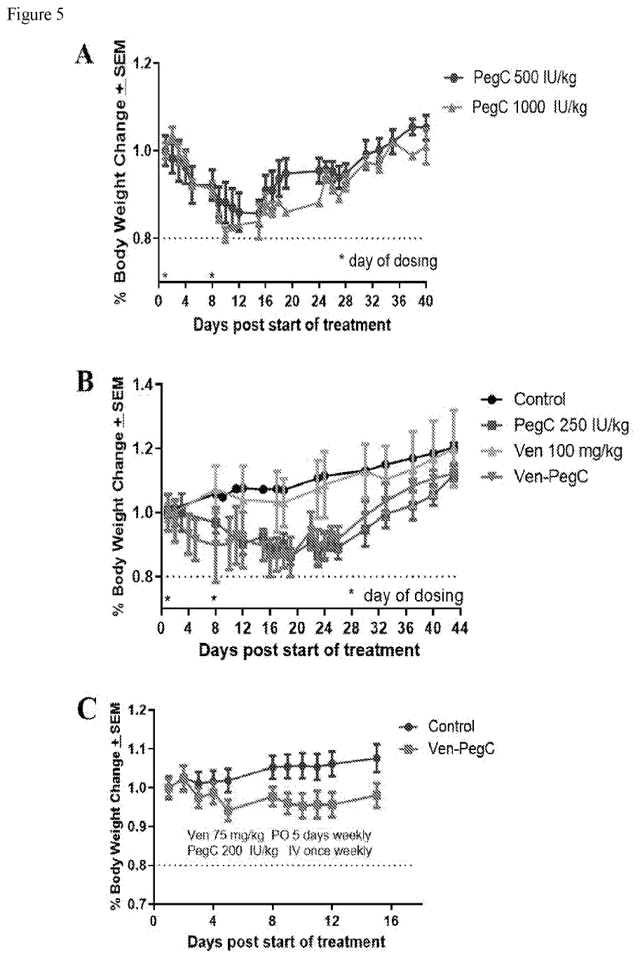

After demonstrating Ven-PegC's in vitro efficacy, the tolerability and safety of PegC monotherapy and Ven-PegC was tested in non-leukemia bearing NRG mice. To determine the maximum tolerated dose (MTD) of PegC, single-agent PegC was intravenously (IV) injected once weekly for two weeks at much higher doses than reported in the literature (i.e., 1000 and 500 IU/kg) [33]. Mice lost ˜20% body weight after these very high doses of single-agent PegC, and fatalities were observed (FIG. 5A). The MTD for PegC monotherapy was determined to be 250 IU/kg. To determine the MTD of the Ven-PegC, NRG mice were treated with 250 IU/kg PegC IV weekly plus a literature-based dosing schedule for Ven [34] of 100 mg/kg PO 5 days per week. In response to significant weight loss in the Ven-PegC group (FIG. 5B) after one week of treatment, the doses of Ven was reduced to 75 mg/kg and PegC to 200 IU/kg with no change in schedule (FIG. 5C).

The effect of Ven-PegC combination treatment was then tested for two weeks on complete blood counts (CBC) as well as on hepatic and renal function, pancreatic enzymes and coagulation markers in immune competent female and male CD1 mice. Weight loss was small and transient (FIG. 5C). Following euthanasia after two weeks, blood and plasma were analyzed. Ven-PegC caused leukopenia, a known adverse event of Ven but not asparaginase (Table 1), but no other blood cell count changes [2,35]. Transaminases, bilirubin, amylase and lipase were not elevated in the Ven-PegC group, indicating no hepatotoxicity or pancreatitis (Table 1), nor were there substantial changes in fibrinogen and prothrombin time (PT) values (Table 1). Similarly, the combination of Ven plus a different asparaginase (E. Coli pegaspargase) caused no changes in blood cell counts or comprehensive metabolic panel (CMP) [36] (data not shown).

TABLE 1
Effect of Ven-PegC on blood counts
and organ function of interest

| | Units | Control | Ven-PegC | P-Value |
|---|---|---|---|---|
| WBC | K/μL | 5.7 ± 1.2 | 1.3 ± 0.4 | 0.04 |
| RBC | M/μL | 7.9 ± 0.4 | 7.0 ± 0.7 | NS |
| HGB | g/dL | 12.2 ± 0.5 | 10.5 ± 1.2 | NS |
| Platelets | K/μL | 694 ± 110 | 1092 ± 277 | NS |
| Amylase | U/L | 1179 ± 72 | 1055 ± 71 | NS |
| Lipase | U/L | 85.3 ± 7.9 | 60.8 ± 5.8 | 0.05 |
| Glucose | mg/dL | 263 ± 32 | 213 ± 22 | NS |
| BUN | mg/dL | 24.5 ± 1.9 | 22 ± 1.3 | NS |
| Creatinine | mg/dL | 0.31 ± 0.03 | 0.28 ± 0.02 | NS |
| Fibrinogen | mg/dL | 173 ± 1.7 | 153.9 ± 1.5 | 0.0003 |
| ALT | U/L | 121.4 ± 46.9 | 83.3 ± 20.4 | NS |
| AST | U/L | 200.8 ± 66.7 | 382 ± 196.5 | NS |
| Total Bilirubin | mg/dL | 0.36 ± 0.04 | 0.37 ± 0.02 | NS |
| Prothrombin | sec | 33.6 ± 2.0 | 12.5 ± 2.0 | 0.002 |
| Time (PT) | | | | |

NS = Not Statistically Significant

### Example 6—Combination of PegC and Ven Demonstrates Potent Anti-AML Efficacy In Vivo

To test the anti-AML efficacy of Ven-PegC combination, the AML45-luc PDX model was selected, consisting of luciferase-expressing primary cells cryopreserved from a patient with relapsed AML harboring a complex karyotype which had transformed from myelodysplastic syndrome (MDS). NRG mice transplanted with AML45-luc cells were imaged 3-10 days post-transplant to assess AML45 burdens and to confirm engraftment, then groups were treated with vehicle, Ven, PegC or Ven-PegC (FIG. 6). Serial weekly imaging quantitations of AML burden demonstrated a major decrease in AML burden only in the Ven-PegC group. By Day 36, no leukemia was detected in mice treated with Ven-PegC, whereas all of the other mice had massive leukemia burdens (FIG. 6A). Treatment was suspended during Weeks 5-11, and then reinitiated for a final three weeks (Days 78-103).

By quantitation of AML45-luc luminescence via in vivo imaging, the Ven-treated (p=0.006) and PegC-treated (p=0.001) groups of mice had slightly lower photon intensity (leukemia burdens) than the vehicle control group. However, the mice in Ven-PegC group had substantially lower leukemia burdens (p<0.0001) (FIG. 6B). One mouse in the Ven-PegC group developed significant AML45-luc luminescence over time, but that occurred late, after Day 100. No mouse in the Ven-PegC group reached a critical leukemia burden (i.e., AML45-luc luminescence photon intensity >1.5×10⁸), which was uniformly followed rapidly by death) due to AML.

Overall survival results were consistent with those of tumor burden. All mice in the vehicle control group died by Day 99 with massive leukemia burdens. Mice treated with Ven-PegC lived significantly longer than all other mice (log rank p<0.0001) (FIG. 6C). Ven-PegC treated mice had transient early weight loss, but recovered and gained weight. Notably, re-treatment at week 11 caused no additional weight loss (FIG. 6D).

To confirm the robust efficacy of Ven-PegC observed against the AML45-luc xenograft model, this combination was evaluated in U937-luc, an extremely aggressive orthotopic in vivo model of acute monocytic leukemia with a complex karyotype. Post engraftment, U937-luc-bearing NRG mice were treated with vehicle, Ven, PegC, Ven-PegC, azacitidine (Aza), or the FDA-approved Aza-Ven combination. After one week of dosing, leukemia burden by U937-luc bioluminescence had increased markedly in the Aza and Aza-Ven treated groups, but not the Ven-PegC-treated, or to a lesser extent, PegC-treated groups (FIG. 7A, 7B; p<0.001 for Ven-PegC and PegC vs control). All mice in the vehicle-treated, Ven-treated, Aza-treated, and Aza-Ven-treated groups died by Day 18 (FIG. 7A-C), while survival was significantly prolonged in groups treated with PegC or Ven-PegC.

### Example 7—Pharmacodynamics: PegC Effectively Depletes Plasma Glutamine and Modulates Translation-Associated Molecules In Vivo

Another in vivo study was performed to confirm again the efficacy of Ven-PegC for the third time and to evaluate the in vivo pharmacodynamics. In this experiment, a second cohort of mice engrafted with AML45-luc cells was treated with vehicle, Ven, PegC or Ven-PegC and the experiment was terminated on Day 39 in all animals to collect blood and tissue for pharmacodynamics (PD) assays. Ven-PegC again showed clear superiority to the other treatment groups (FIG. 8A and 8B). On Day 39 post start of treatment, mice were euthanized, blood was drawn and plasma was isolated for amino acid analysis, and bone marrow cells were harvested for immunoblot and qRT-PCR analyses.

In the PegC-treated and Ven-PegC-treated compared to vehicle-treated groups, plasma Glutamine (p=0.0001) and Asparagine (p=0.0001) were completely depleted (FIG. 8C) and plasma glutamate (Glu) levels were increased significantly (p=0.0006) (FIG. 8C). While the comprehensive plasma amino acid analysis from all treated mice showed 2-3-fold differences in levels of a few other amino acids, e.g. serine and glycine, but the magnitudes of changes of Glutamine, Asparagine, and Glutamate were far greater (data not shown). No significant differences in amino acid levels were noted in Ven-treated mice (data not shown).

To test the in vivo treatment effect on the expression of proteins identified in the mechanistic studies, protein lysates were prepared from isolated bone marrow cells of mice post-treatment and tested by western analysis. Consistent with the in vitro results on molecules involved in the translational phase of protein synthesis, significantly lower levels of p90RSK and phosphorylation of p70S6K, 4EBP1, ERK and eIF4E was observed in the bone marrow cells of Ven-PegC-treated mice (FIG. 8E). In addition, expression of MCL-1, but not BCL2 or BCL-XL, protein was decreased substantially in Ven-PegC-treated mice (FIG. 8D). Consistent with the transcriptome profile, mRNA expression of PegC, Ven and Ven-PegC signature genes was modulated in treated PDX mice (data not shown). Furthermore, the protein levels of translatome-specific genes (data not shown), but not their mRNA levels (data not shown), were noted to be modulated similarly to the in vitro observations. These pharmacodynamic results confirm effective inhibition of mRNA translation and cellular protein synthesis as the mechanism of anti-leukemic action of Ven-PegC in vivo.

### Example 8—Human Clinical Trial on Glutamine Depletion from Plasma

An investigator-initiated clinical trial was conduct on human subjects having AML. The purpose the Phase 1 clinical trial was, in part, to demonstrate that asparaginase with achievable plasma activity ≥0.1 IU/mL is able to effectively deplete plasma glutamine to undetectable levels in patients with R/R AML. The lowest threshold for glutamine detection in the mass spectrometry assay was 12.5 μmol/L. The ability of Erwinia asparaginase to decrease plasma glutamine levels to ≤120 μmol/L with acceptable safety profile was the “primary objective” of the Phase 1 study. Plasma glutamine levels were measured 48 hours after the first dose and immediately before each subsequent dose (i.e. trough level) of Erwinia asparaginase administered on three times weekly for two consecutive weeks (days 1, 3, 5, 8, 10, and 12). FIG. 10 shows the results of trough plasma glutamine levels in the five patients with AML who were enrolled in the study. Plasma glutamine measured immediately before administration of short acting Erwinia asparaginase (48-72 hours after administration of the previous dose) was considered trough glutamine level.

The preliminary data suggest that patients, whose plasma glutamine levels became undetectable, had higher probability of achieving clinical response/remission. For example, Patient 3, a 72 years old man, whose AML with normal karyotype with IDH1 mutation was refractory to decitabine, responded to one cycle of single agent asparaginase as evidenced by Day +30 bone marrow biopsy showing a decrease in the bone marrow blasts from 46% to 12% with a meaningful clinical benefit of platelet transfusion independency (platelet count of 109,000/μL). Patient 4, an 84 years old man, with AML-M6 (erythroleukemia) with trisomy 21 and DNMT3A, PTPN11, RUNX1, and SR3B1 mutations, whose AML relapsed after several cycles of decitabine, responded to one cycle of asparaginase—Day +29 CBC showed no myeloblasts in peripheral blood, absolute neutrophil count of 530/mcL, platelet counts of 15-20 K/mcL (his baseline), and a decrease in bone marrow blasts from 30% to 15%. Both remained alive with robust performance status for more than a year. AML in Patient number 2 progressed rapidly and she succumbed to her disease. She was the only patient in whom plasma glutamine level did not decrease. From safety stand point, no dose limiting toxicity (DLT) was observed in any of the five patients.

While the invention has been described with reference to certain particular embodiments thereof, those skilled in the art will appreciate that various modifications may be made without departing from the spirit and scope of the invention. The scope of the appended claims is not to be limited to the specific embodiments described.

### REFERENCES

All patents and publications mentioned in this specification are indicative of the level of skill of those skilled in the art to which the invention pertains. Each cited patent and publication is incorporated herein by reference in its entirety. All of the following references have been cited in this application:
1. A. J. Souers et al., ABT-199, a potent and selective BCL-2 inhibitor, achieves antitumor activity while sparing platelets. Nature medicine 19, 202-208 (2013).
2. FDA, Venetoclax (VENCLEXTA). Food and Drug Administration, https://www.accessdata.fda.gov/drugsatfda_docs/label/2019/208573s2080131bl.pdf (2018).
3. C. D. DiNardo et al., Venetoclax combined with decitabine or azacitidine in treatment-naive, elderly patients with acute myeloid leukemia. Blood 133, 7-17 (2019).
4. A. C. Winters et al., Real-world experience of venetoclax with azacitidine for untreated patients with acute myeloid leukemia. Blood Adv 3, 2911-2919 (2019).
5. M. Konopleva et al., Efficacy and Biological Correlates of Response in a Phase II Study of Venetoclax Monotherapy in Patients with Acute Myelogenous Leukemia. Cancer Discov 6, 1106-1117 (2016).
6. C. D. DiNardo et al., Clinical experience with the BCL2-inhibitor venetoclax in combination therapy for relapsed and refractory acute myeloid leukemia and related myeloid malignancies. American journal of hematology 93, 401-407 (2018).
7. N. Jacque et al., Targeting glutaminolysis has antileukemic activity in acute myeloid leukemia and synergizes with BCL-2 inhibition. Blood 126, 1346-1356 (2015).
8. R. Bajpai et al., Targeting glutamine metabolism in multiple myeloma enhances BIM binding to BCL-2 eliciting synthetic lethality to venetoclax. Oncogene 35, 3955-3964 (2016).
9. L. Willems et al., Inhibiting glutamine uptake represents an attractive new strategy for treating acute myeloid leukemia. Blood 122, 3521-3532 (2013).
10. A. Emadi et al., Glutaminase inhibition selectively slows the growth of primary acute myeloid leukemia (AML) cells with isocitrate dehydrogenase (IDH) mutation. Blood (ASH Annual Meeting Abstracts) 120, 2624 (2012).
11. A. Beckett, D. Gervais, What makes a good new therapeutic L-asparaginase? World J Microbiol Biotechnol 35, 152 (2019).
12. Z. B. Moola, M. D. Scawen, T. Atkinson, D. J. Nicholls, Erwinia chrysanthemi L-asparaginase: epitope mapping and production of antigenically modified enzymes. The Biochemical journal 302 (Pt 3), 921-927 (1994).
13. A. Emadi, H. Zokaee, E. A. Sausville, Asparaginase in the treatment of non-ALL hematologic malignancies. Cancer chemotherapy and pharmacology 73, 875-883 (2014).
14. A. Emadi et al., Asparaginase Erwinia chrysanthemi effectively depletes plasma glutamine in adult patients with relapsed/refractory acute myeloid leukemia. Cancer chemotherapy and pharmacology 81, 217-222 (2018).
15. R. E. Rau et al., Outcome of pediatric patients with acute lymphoblastic leukemia/lymphoblastic lymphoma with hypersensitivity to pegaspargase treated with PEGylated Erwinia asparaginase, pegcrisantaspase: A report from the Children's Oncology Group. Pediatric Blood & Cancer 65(3), 1-14 (2018).
16. H. A. Nguyen et al., A Novel 1-Asparaginase with low 1-Glutaminase Coactivity Is Highly Efficacious against Both T- and B-cell Acute Lymphoblastic Leukemias In Vivo. Cancer research 78, 1549-1560 (2018).
17. I. M. van der Sluis et al., Consensus expert recommendations for identification and management of asparaginase hypersensitivity and silent inactivation. Haematologica 101, 279-285 (2016).
18. T. C. Chou, P. Talalay, Quantitative analysis of dose-effect relationships: the combined effects of multiple drugs or enzyme inhibitors. Adv Enzyme Regul 22, 27-55 (1984).
19. C. L. Lomelino, J. T. Andring, R. McKenna, M. S. Kilberg, Asparagine synthetase: Function, structure, and role in disease. J Biol Chem 292, 19952-19958 (2017).
20. L. Lin, S. A. White, K. Hu, Role of p90RSK in Kidney and Other Diseases. Int J Mol Sci 20(4), 972, 1-13 (2019).
21. F. Angenstein, W. T. Greenough, I. J. Weiler, Metabotropic glutamate receptor-initiated translocation of protein kinase p90rsk to polyribosomes: a possible factor regulating synaptic protein synthesis. Proceedings of the National Academy of Sciences of the United States of America 95, 15078-15083 (1998).
22. M. J. Boucher et al., MEK/ERK signaling pathway regulates the expression of Bcl-2, Bcl-X(L), and Mcl-1 and promotes survival of human pancreatic cancer cells. J Cell Biochem 79, 355-369 (2000).
23. A. N. Hata, J. A. Engelman, A. C. Faber, The BCL2 Family: Key Mediators of the Apoptotic Response to Targeted Anticancer Therapeutics. Cancer Discov 5, 475-487 (2015).
24. M. Subramanian, C. Shaha, Up-regulation of Bcl-2 through ERK phosphorylation is associated with human macrophage survival in an estrogen microenvironment. Journal of immunology 179, 2330-2338 (2007).
25. S. M. Kornblau et al., Simultaneous activation of multiple signal transduction pathways confers poor prognosis in acute myelogenous leukemia. Blood 108, 2358-2365 (2006).
26. S. Elf et al., p90RSK2 is essential for FLT3-ITD- but dispensable for BCR-ABL-induced myeloid leukemia. Blood 117, 6885-6894 (2011).
27. G. S. Choudhary et al., MCL-1 and BCL-xL-dependent resistance to the BCL-2 inhibitor ABT-199 can be overcome by preventing PI3K/AKT/mTOR activation in lymphoid malignancies. Cell death & disease 6, e1593 (2015).
28. Q. Wang, J. Wan, W. Zhang, S. Hao, MCL-1 or BCL-xL-dependent resistance to the BCL-2 antagonist (ABT-199) can be overcome by specific inhibitor as single agents and in combination with ABT-199 in acute myeloid leukemia cells. Leuk Lymphoma, 1-11 (2019).
29. Y. Iiboshi, P. J. Papst, S. P. Hunger, N. Terada, L-Asparaginase inhibits the rapamycin-targeted signaling pathway. Biochem Biophys Res Commun 260, 534-539 (1999).
30. N. Hay, Mnk earmarks eIF4E for cancer therapy. Proceedings of the National Academy of Sciences of the United States of America 107, 13975-13976 (2010).
31. B. Kapadia et al., Fatty Acid Synthase induced S6Kinase facilitates USP11-eIF4B complex formation for sustained oncogenic translation in DLBCL. Nature communications 9, 829 (2018).
32. R. Bourgon, R. Gentleman, W. Huber, Independent filtering increases detection power for high-throughput experiments. Proceedings of the National Academy of Sciences of the United States of America 107, 9546-9551 (2010).
33. W. W. Chien et al., Pharmacology, immunogenicity, and efficacy of a novel pegylated recombinant Erwinia chrysanthemi-derived L-asparaginase. Investigational new drugs 32, 795-805 (2014).
34. E. D. V. Campos, R. Pinto, Targeted therapy with a selective BCL-2 inhibitor in older patients with acute myeloid leukemia. Hematol Transfus Cell Ther 41, 169-177 (2019).
35. A. Emadi, N. A. Bade, B. Stevenson, Z. Singh, Minimally-Myelosuppressive Asparaginase-Containing Induction Regimen for Treatment of a Jehovah's Witness with mutant IDH1/NPM1/NRAS Acute Myeloid Leukemia. Pharmaceuticals (Basel) 9(1), 1-7 (2016).
36. N. A. Bade et al., Optimizing pegylated asparaginase use: An institutional guideline for dosing, monitoring, and management. J Oncol Pharm Pract, 1078155219838316 (2019).
37. M. L. Slovak et al., Karyotypic analysis predicts outcome of preremission and postremission therapy in adult acute myeloid leukemia: a Southwest Oncology Group/Eastern Cooperative Oncology Group Study. Blood 96, 4075-4083 (2000).
38. J. C. Byrd et al., Pretreatment cytogenetic abnormalities are predictive of induction success, cumulative incidence of relapse, and overall survival in adult patients with de novo acute myeloid leukemia: results from Cancer and Leukemia Group B (CALGB 8461). Blood 100, 4325-4336 (2002).
39. S. O. Ciurea et al., Relapse and survival after transplantation for complex karyotype acute myeloid leukemia: A report from the Acute Leukemia Working Party of the European Society for Blood and Marrow Transplantation and the University of Texas MD Anderson Cancer Center. Cancer 124, 2134-2141 (2018).
40. J. Bodo et al., Acquired resistance to venetoclax (ABT-199) in t(14;18) positive lymphoma cells. Oncotarget 7, 70000-70010 (2016).
41. S. K. Tahir et al., Potential mechanisms of resistance to venetoclax and strategies to circumvent it. BMC Cancer 17, 399 (2017).
42. J. Deng et al., BH3 profiling identifies three distinct classes of apoptotic blocks to predict response to ABT-737 and conventional chemotherapeutic agents. Cancer Cell 12, 171-185 (2007).
43. D. Yecies, N. E. Carlson, J. Deng, A. Letai, Acquired resistance to ABT-737 in lymphoma cells that up-regulate MCL-1 and BFL-1. Blood 115, 3304-3313 (2010).
44. R. Guieze et al., Mitochondrial Reprogramming Underlies Resistance to BCL-2 Inhibition in Lymphoid Malignancies. Cancer Cell 36, 369-384 e313 (2019).
45. J. T. Opferman et al., Obligate role of anti-apoptotic MCL-1 in the survival of hematopoietic stem cells. Science 307, 1101-1104 (2005).
46. B. Vick et al., Knockout of myeloid cell leukemia-1 induces liver damage and increases apoptosis susceptibility of murine hepatocytes. Hepatology 49, 627-636 (2009).
47. R. L. Thomas et al., Loss of MCL-1 leads to impaired autophagy and rapid development of heart failure. Genes & development 27, 1365-1377 (2013).
48. A. J. Bott et al., Oncogenic Myc Induces Expression of Glutamine Synthetase through Promoter Demethylation. Cell metabolism 22, 1068-1077 (2015).
49. A. Emadi, Exploiting AML vulnerability: glutamine dependency. Blood 126, 1269-1270 (2015).
50. H. G. Wendel et al., Dissecting eIF4E action in tumorigenesis. Genes & Development 21, 3232-3237 (2007).
51. R. Tang et al., Semisynthetic homoharringtonine induces apoptosis via inhibition of protein synthesis and triggers rapid myeloid cell leukemia-1 down-regulation in myeloid leukemia cells. Mol Cancer Ther 5, 723-731 (2006).
52. A. C. Shetty et al., CAVERN: Computational and visualization environment for RNA-seq analyses. 69th Annual Meeting American Society of Human Genetics (2019).
53. S. Andrews, FastQC A Quality Control tool for High Throughput Sequence Data [Online]. http ://www.bioinformati cs.babraham.ac.uk/proj ects/fastqc/ [Accessed: 9 Oct. 2015], (2010).
54. D. Kim, B. Langmead, S. L. Salzberg, HISAT: a fast spliced aligner with low memory requirements. Nat Methods 12, 357-360 (2015).
55. S. Anders, P. T. Pyl, W. Huber, HTSeq-a Python framework to work with high-throughput sequencing data. Bioinformatics 31, 166-169 (2015).

56. S. Anders, W. Huber, Differential expression analysis for sequence count data. Genome Biol 11, R106 (2010).

1. A method of treating cancer in a subject or prolonging survival of a subject having cancer, comprising administering therapeutically effective amounts of a first agent that depletes plasma glutamine and a second agent that inhibits BCL-2 activity to a subject having cancer.

2. The method of claim 1, wherein the first and second agents are administered in any order, alone or in any combination, sequentially or concurrently, with overlapping or non-overlapping periods of administration.

3. The method of claim 2, comprising concurrently administering therapeutically effective amounts of the first and second agents.

4. The method of claim 2, comprising sequentially administering therapeutically effective amounts of the first and second agents, in any order.

5. The method of claim 1, wherein one or more of the first and second agents are formulated, separately or together, in any combination, in pharmaceutical compositions comprising a pharmaceutically acceptable carrier or diluent.

6. The method of claim 1, wherein the combination of the first and second agents has an additive therapeutic effect on the cancer. (original) The method of claim 1, wherein the combination of the first and second agents has a synergistic therapeutic effect on the cancer.

8. The method of claim 1, wherein the agent that depletes plasma glutamine is one or more asparaginase selected from the group consisting of E. coli-derived short acting asparaginase, polyethylene glycosylated E. coli-derived asparaginase (pegaspargase or calaspargase pegol-mknl), Erwinia chrysanthemi-derived short acting asparaginase (Erwinaze), Erwinia chrysanthemi-derived short acting asparaginase (crisantaspase), polyethylene glycosylated Erwinia chrysanthemi-derived asparaginase (pegcrisantaspase; PegC).

9. The method of claim 1, wherein the therapeutically effective amount of the agent that depletes plasma glutamine is between about 10 and about 50,000 IU/m2.

10. The method of claim 1, wherein the agent that inhibits BCL-2 activity is one or more of Venetoclax (Ven), BCL201, and navitoclax.

11. The method of claim 1, wherein the therapeutically effective amount of the agent that inhibits BCL-2 activity is between about 10 and about 800 mg.

12. The method of claim 1, wherein the cancer is one or more cancers selected from the consisting of acute myeloid leukemia (AML), complex karyotype acute myeloid leukemia (CK-AML), acute lymphoblastic leukemia (ALL), B cell ALL (B-ALL), T cell ALL (T-ALL), chronic myeloid leukemia (CIVIL), chronic lymphoid leukemia (CLL); lymphoma (including B cell and T cell); myeloma; myelodysplastic syndrome; non-small cell lung cancer; pancreatic cancer; gastric cancer; Kaposi's sarcoma; hepatocellular carcinoma; osteosarcoma; laryngeal squamous cell carcinoma; metastatic uveal melanoma; lung and splenic metastases; advanced non-small cell lung cancer; cervical carcinoma; colorectal cancer; breast cancer; prostate cancer; mesothelioma; and all other hematologic malignancies and solid cancers including brain cancers.

13. A method of treating AML in a subject, comprising administering therapeutically effective amounts of an asparaginase and Ven to a subject having AML.

14-15. (canceled)

16. A method of prolonging survival of a subject having AML, comprising administering therapeutically effective amounts of an asparaginase and Ven to a subject having AML.

17-18. (canceled)

19. The method of claim 1, wherein the agent that depletes plasma glutamine is PegC.

20. The method of claim 1, wherein the agent that inhibits BCL-2 activity is Ven.

21. The method of claim 13, wherein the asparaginase and Ven are administered in either order, alone or in combination, sequentially or concurrently, with overlapping or non-overlapping periods of administration.

22. The method of claim 16, wherein the asparaginase and Ven are administered in either order, alone or in combination, sequentially or concurrently, with overlapping or non-overlapping periods of administration.
